SEQUENCE LISTING (2 sequences)

SEQ ID NO: 1 - protein, 16 aa, Artificial Sequence
  Synthetic Construct
XXXXXSXXXXXXXXXX

SEQ ID NO: 2 - protein, 10 aa, Artificial Sequence
  Synthetic construct
MAIPKIASYP

(19) United States
(12) Patent Application Publication
(10) Pub. No.: US 2006/0216775 A1
(43) Pub. Date: Sep. 28, 2006

(54) Compositions and methods for analysis and manipulation of enzymes in biosynthetic proteomes

(75) Inventors: Michael D. Burkart, San Diego, CA (US); James J. Laclair, San Diego, CA (US)

Correspondence Address: WOODCOCK WASHBURN LLP, ONE LIBERTY PLACE, 46TH FLOOR, 1650 MARKET STREET, PHILADELPHIA, PA, 19103, US

(73) Assignee: The Regents of the University of Califoenia, Oakland, CA (US)

(21) Appl. No.: 10/561,108

(22) PCT Filed: Jun. 17, 2004
(86) PCT No.: PCT/US04/19568

§ 371 Date: May 11, 2006

Related U.S. Application Data
(60) Provisional application No. 60/479,344, filed on Jun. 17, 2003.

(51) Int. Cl. C40B 30/02; C12Q 1/48; G06F 19/00

(52) U.S. Cl. 435/15; 702/19

(57) ABSTRACT

This invention generally relates to methods and compositions for identifying a protein of interest. The compositions further provide microarrays. The methods provide a viable screen for genetic and proteornic events in natural and engineered systems.

### CROSS-REFERENCE TO RELATED APPLICATION

This application claims priority to U.S. Application No. 60/479,344, filed Jun. 17, 2003, the entire disclosure of which is incorporated herein by reference.

### FIELD

This invention generally relates to methods and compositions for identifying biosynthetic enzymes involved in secondary metabolic biosynthesis or other proteins of interest. The compositions and methods provide microarray analysis and provide a screen for genetic and proteomic events in natural and engineered systems.

### BACKGROUND

Fatty acid (FA), polyketide (PK) and non-ribosomal peptide (NRP) biosyntheses have been elucidated through a four-stage process. The first stage serves to isolate and identify natural molecules and screen their biological activity. Research at this stage has led to the discovery of a large number of bioactive natural products. Continuing research primarily focuses on marine organisms and involves organism collection, bioassay screening, natural product isolation, and structure elucidation.

The second stage serves to elucidate the biosynthetic pathway(s) from a producer organism. This stage entails the isolation and sequencing of the genes involved in a natural biosynthesis. In order to complete a sequencing inquiry, researchers must definitively demonstrate activity of at least one enzyme in a biosynthetic cluster, either through knockout experiments that alter molecular structure or through in vitro proof of activity. Complicating this research is the abundance of non-culturable microorganisms that produce interesting bioactive molecules. For instance, many natural products isolated from marine organisms such as sea cucumbers apparently arise from unculturable symbiotic bacteria. On a different but similar issue, many organismic strains produce bioactive molecule through uncharted biosynthetic pathways.

Once a biosynthetic pathway has been fully sequenced, the activity, order, and timing of each enzyme in its pathway must be determined. Each gene product usually corresponds to an enzymatic step in the biosynthesis. This union between gene and enzyme must be determined and demonstrated. Here, one can often draw analogies from previously studied enzymes through protein sequence similarity and/or homology, thereby identifying parallels with other known secondary metabolism pathways. In many cases, each enzyme is produced individually and activity studies are performed in vitro to validate a proposed pathway. Alternatively, the pathway may be studied genetically by generating mutants of the producer organism in which the products of an individual gene has been altered, thereby producing pathway intermediates that may be correlated with the missing enzyme. Often combinations of these techniques lead to a complete understanding of enzyme activity. Gene sequence within a given pathway does not necessarily correspond to sequential enzyme activity, and the order of events must also be correlated to enzyme function in order to fully understand metabolite construction.

Using techniques of molecular biology, genes for biosynthetic enzymes of interest from characterized biosynthetic pathways are assembled into heterologous hosts. Difficulties within this process relate to the fact that there are very few rules, and only a few natural product pathways have yet been engineered. Non-ribosomal peptide (NRP), polyketide (PK), carbohydrate, terpene, sterol, shikimic acid, and fatty acid pathways are all of interest to current researchers. Most heterologous host organisms to date have been chosen from a set of easily manipulable bacteria, typically Escherichia coli. Once a new pathway has been created, mathematical models of metabolite flux are studied to determine optimum fermentative output and minimum growth requirements. New genetic tools, including gene promoters, repressors, and signaling pathways, are continually being developed and optimized for applications to metabolic engineering.

The biosynthesis of natural products derived from fatty acid (FA), polyketide (PK) and non-ribosomal peptide (NRP) origins have been of great interest recently in both drug discovery and production arenas. Recently, genetic approaches have also provided effective entry into the recombinant isolation of biosynthetic processes. In the latter approach, genomic DNA or DNA engineered thereon is transformed into a suitable host organism. Incorporation and translation of the foreign DNA within this host, serves to recreate a given biosynthesis. Upon developing culturing conditions, the biosynthesis is elucidated by the combination of genetic and protein studies. Currently, genetic approaches are commonly used to sequence, clone and purify proteins involved in FA, PK and NRP biosynthesis. Cane D. E., et al., Chem. Biol., 6: R319-R325; Du L., et al., Curr. Opin. Drug Discov. Devel., 4: 215-228, 2001; Doekel S., et al., Metab. Eng. 3: 64-77, 2001; Cane D. E., et al., Science, 282: 63-68, 1998; Strohl W. R., Metab. Eng., 3: 4-14, 2001; Du L., et al., Metab. Eng. 3: 78-95, 2001; Fenical W., Trends Biotechnol., 15: 339-341, 1997; Courtois S., et al., Appl. Environ. Microbiol., 69, 49-55, 2003; Nielsen J., Curr. Opin. Microbiol., 1: 330-336, 1998; Hutchinson C. R., Curr. Opin. Microbiol., 1: 319-329, 1998; Kao C. M., et al., Science, 265: 509-512, 1994; Bedford D. J., et al., J. Bacteriol., 177: 4544-4548, 1995; Kim E. S., et al., J. Bacteriol., 177: 1202-1207, 1995; Kealey J. T., et al., Proc. Natl. Acad. Sci. USA., 95: 505-509, 1998.

Since elucidation of the modular nature of their biosynthetic machinery, FA, PK and NRP synthases have been aggressively studied for the potential of engineering their structure toward directed or combinatorial biosynthesis, while at the same time there exists widespread optimism that novel drugs of these molecular classes will continue to be discovered in nature. Compositions and methods for identification and manipulation of these synthases prove useful in the discovery of new biosynthetic systems and as analytical tools for engineered systems. Here compositions and methods for the tagging FA, PK and NRP synthases with fluorescent and biotin-linked probes in a selective manner is defined. Analysis and purification of the tagged proteins are subsequently performed with SDS-PAGE electrophoresis, blot analysis, and affinity chromatography. These tools can be used to selectively manipulate biosynthetic enzymes from recombinant and natural producer organisms for the purpose of detecting protein expression, solubility, and activity. Pfeifer B. A., et al., Microbiol. Mol. Biol. Rev., 65: 106-118, 2001.

The identification and isolation of FA, PK and NRP gene systems is a relatively new goal of natural product scientists. Recent genomic approaches have addressed situations in which an organism possesses multiple genes for antibiotic biosynthesis but does not produce the small molecules in significant quantities for isolation and identification. Similar circumstances have been identified in some human pathogens known to produce PK virulence factors, yet these molecules have not been isolated. Until recently, identification of new NRP and PK biosynthetic enzymes necessarily followed isolation of small molecules in a native producer. Only when a small molecule showed significant therapeutic potential or interesting structure would researchers delve into its biosynthesis. Lambalot R. H., et al., Chem. Biol., 3: 923-936, 1996; Quadri L. E., et al., Biochemistry, 37: 1585-1595, 1998; Mofid M. R., et al., J. Biol. Chem., 277: 17023-17031, 2002; Belshaw P. J., et al., Science, 284: 486-489, 1999.

Often, the genes responsible for small molecule biosynthesis remain elusive to sequencing efforts. Homologous DNA sequences, a key for identifying NRP and PK synthase coding domains, can also serve to mask one biosynthetic system from others. This complication often requires lengthy cosmid library construction and gene probing experiments.

Due to difficulties in culturing and metabolite overproduction in natural producer strains such as actinomycetes, bacilli, and filamentous fungi, continuing efforts have focused on the heterologous expression of biosynthetic clusters in host organisms more amenable to laboratory manipulation and industrial culturing, particularly Streptomyces coelicolor and more recently E. coli. PK/NRP biosynthetic enzymes are difficult to express heterologously for several reasons. First, they are large enzymes, usually ranging in molecular weights between 300-800 kDa. Their sheer size presents a major obstacle to their routine cloning and manipulation. Second, the majority of large megasynthase proteins heterologously expressed in E. coli either form insoluble aggregates or show no activity in soluble form. Additionally, the genomes of actinomycetes, a source of many PK/NRP biosynthetic genes, are guanine and cytosine (GC) rich, presenting difficulties for in vitro experiments like PCR.

### SUMMARY

The methods and compositions described herein are applicable to screen for elements of fatty acid (FA), polyketide (PK) and non-ribosomal peptide (NRP) synthesis. These methods and compositions are applicable to the study of all stages of FA, PKS and NRP biosynthesis. These methods and compositions provide an entry to a proteomic system for biosynthetic screening by providing the tools necessary to screen for biosynthetic enzymes and proteins, and verify and quantify activity within metabolically engineered systems. Using recombinant DNA and molecular genetic methods, carrier protein domains can be cloned in fusion with any protein of interest. The resulting fusion system thereby allows the methods and compositions of the present invention to be extended to the study of any protein of interest, whereby carrier protein analysis is conducted as an C-terminal, N-terminal or internally fused peptide.

In one embodiment, a method for detecting a protein of interest is provided comprising contacting a coenzyme with a synthetic appendage label, contacting a carrier protein domain with the protein of interest to form a carrier protein (CP) domain-protein of interest (POI) complex, contacting the carrier protein (CP) domain-protein of interest (POI) complex with the labeled coenzyme to form a labeled coenzyme-carrier protein (CP) domain-protein of interest (POI) complex, and detecting the labeled carrier protein domain to detect the protein of interest.

In a detailed aspect the the CP domain is a biosynthetic enzyme carrier protein domain. In a further detailed aspect, the carrier protein domain is a polyketide (PK) synthase carrier protein domain, a non-ribosomal peptide (NRP) synthase carrier protein domain, or a fatty acid (FA) synthase carrier protein domain. In a further detailed aspect, the polyketide (PK) synthase carrier protein domain comprises at least one domain with acyl carrier protein (ACP) activity. In a further detailed aspect, the non-ribosomal peptide (NRP) synthase carrier protein domain comprises at least one domain with peptidyl carrier protein (PCP), aryl carrier protein (ArCP) and/or acyl carrier protein (ACP) activity. In a further detailed aspect, the fatty acid (FA) synthase carrier protein domain comprises at least one domain with acyl carrier protein (ACP) activity.

In a detailed aspect, the biosynthetic enzyme is a hybrid between a FA synthase, PK synthase, and/or NRP synthase and further comprises at least one domain with acyl carrier protein (ACP) and/or aryl carrier protein (ArCP) activity. In a detailed embodiment, the method further comprises digesting the biosynthetic enzyme with a protease.

In a detailed embodiment, the synthetic appendage label further comprises a linker and a reporter. In a detailed aspect, the reporter is an affinity reporter, a colored reporter, a fluorescent reporter, a magnetic reporter, a radioisotopic reporter, a peptide reporter, a metal reporter, a nucleic acid reporter, a lipid reporter, a glycosylation reporter, or a reactive reporter. In a further detailed aspect, the synthetic appendage label further comprises a protein chip immobilization label, a two-hybrid or three-hybrid analysis label, or a trace purification label. In a further detailed aspect, the reporter is a precursor to an affinity reporter, a colored reporter, a fluorescent reporter, a magnetic reporter, a radioisotopic reporter, a peptide reporter, a metal reporter, a nucleic acid reporter, a lipid reporter, a glycosylation reporter, or a reactive reporter.

In a detailed aspect, the synthetic appendage label contains a linker that unites the thiol terminus of Coenzyme A to an affinity reporter, a colored reporter, a fluorescent reporter, a magnetic reporter, radioactive reporter, or a reactive reporter. In a further detailed aspect, the synthetic reporterappendage reporter contains a precursor to a reporter selected from an affinity reporter, a colored reporter, a fluorescent reporter, a magnetic reporter or a reactive reporter.

In a detailed aspect, carrier proteins and peptides constructed from the carrier proteins can be inserted in fusion with a protein of interest using recombinant genetic methods. The resulting cloned fusion carrier protein can be analysed by treatment with the labeled coenzyme and with the enzyme to form a carrier protein-enzyme-coenzyme complex, transferring the synthetic appendage label from the coenzyme to the carrier protein domain, and detecting the labeled carrier protein domain on the biosynthetic enzyme to identify the biosynthetic enzyme.

In a further detailed embodiment, the method further comprises contacting the labeled coenzyme-carrier protein (CP) domain-protein of interest (POI) complex with a radioactively-labeled coenzyme to form a radioactively labeled coenzyme-carrier protein (CP) domain-protein of interest (POI) complex

In a detailed embodiment the method further comprises contacting the labeled coenzyme-carrier protein (CP) domain-protein of interest (POI) complex with a radioactively-labeled coenzyme to form a radioactively labeled coenzyme-carrier protein (CP) domain-protein of interest (POI) complex.

In a detailed embodiment contacting the carrier protein (CP) domain with the protein of interest (POI) further comprises synthesizing a CP domain-POI fusion protein to form a carrier protein (CP) domain-protein of interest (POI) complex. In a detailed aspect, the carrier protein (CP) domain further comprises an amino acid consensus sequence, [DEQGSTALMKRH]-[LIVMFYSTAC]-[GNQ]-[LIVMFYAG]-[DNEKHS]-S-[LIVMST]-{PCFY }-[STAGCPQLVMF]-[LIVMATN]-[DENQGTAKRHLM]-[LIVMWSTA]-[LIVGSTACR]-(x2)-[LIVMFA].

In a detailed aspect, the labeled coenzyme-CP domain-POI complex further comprises coenzyme A (CoA) or a derivative thereof.

In a detailed embodiment, the method further comprises contacting the CP domain-POI complex and the labeled coenzyme with a phosphotransferase enzyme to form a labeled coenzyme CP domain-POI complex. In a detailed aspect, the phosphotransferase enzyme is a 4′-phosphopantetheinyl transferase. In a further detailed aspect, the method further comprises detecting or modulating a function of label by interaction with a secondary molecule. In a further detailed aspect, the secondary molecule is a carbohydrate, a protein, a peptide, an oligonucleotide, or a synthetic receptor.

In another embodiment, the method further comprises assembling libraries of biosynthetic enzymes, coenzymes and synthetic appendage labels, contacting individual units of biosynthetic enzymes, coenzymes and synthetic appendage labels from libraries of POIs, coenzymes and synthetic appendage labels, and detecting transfer of synthetic appendage label from coenzyme to carrier protein of the biosynthetic enzyme, wherein specificity of the transfer detects the biosynthetic enzyme. In a detailed aspect, the individual units from libraries of coenzymes are spatially-addressed on a three dimensional object. In a further detailed aspect, the individual units from libraries of enzymes are spatially-addressed on a three dimensional object. In a further detailed aspect, the individual units from libraries of labels are spatially-addressed on a three dimensional object. In a further detailed aspect, the individual units from libraries of coenzymes and libraries of enzymes are spatially-addressed on a three dimensional object. In a further detailed aspect, the individual units from libraries of coenzymes and labels are spatially-addressed on a three dimensional object. In a further detailed aspect, the individual units from libraries of coenzymes, labels and enzymes are spatially-addressed on a three dimensional object.

In a further detailed embodiment, the method further comprises identifying the biosynthetic enzyme within a cell culture. In a detailed embodiment the method further comprises identifying the biosynthetic enzyme by molecular weight, wherein the enzyme molecular weight is determined by a technique selected from gel electrophoresis, affinity chromatography or mass spectrometry. In a detailed aspect the method further comprises identifying the protein of interest by nucleic acid or protein sequencing. In a detailed aspect the method further comprises isolating the protein of interest. In a detailed aspect the method further comprises assaying for the expression and/or activity of the protein of interest. In a detailed aspect the method further comprises screening for proteins of interest. In a detailed aspect the method further comprises quantifying the expression a given protein of interest or group of proteins of interest. In a detailed aspect the method further comprises quantifying temporal events related to the expression a given protein of interest.

In a further detailed embodiment, the method further comprises identifying a cell, cell-line, organism or class of organisms characterized by the marking of the protein of interest with the label. In a further detailed embodiment, the method further comprises determining a time of infection or a stage in a cell cycle or a stage in a life cycle. In a detailed aspect the method further comprises determining a level of virulence of the organism. In a detailed aspect the method further comprises identifying novel natural products from the biosynthetic enzyme. In a detailed aspect the method further comprises screening for inhibitors of the biosynthetic pathways. In a detailed aspect the method further comprises measuring individual responses of the biosynthetic enzyme to given conditions to identify the biosynthetic enzyme using a profiler.

In a detailed embodiment, the method further comprises removing chemically or enzymatically the product generated from the transfer of the synthetic appendage label. In a detailed aspect, the method further comprises removing the synthetic appendage label from the carrier protein domain by light. In a detailed aspect, the method further comprises removing the synthetic appendage label from the carrier protein domain by heat. In a detailed aspect, the method further comprises removing the synthetic appendage label from the carrier protein domain by a chemical reagent. In a detailed aspect, the method further comprises removing the synthetic appendage label from the carrier protein domain by an enzyme. In a further detailed aspect, the enzyme is an acyl carrier protein phosphodiesterase.

In another embodiment, a microarray for identification of a protein of interest (POI) comprises a coenzyme linked to a synthetic appendage label, a carrier protein domain contacting the labeled coenzyme and the POI to form a carrier protein-POI-coenzyme complex, the synthetic appendage label transferred from the coenzyme to the carrier protein domain within the microarray, wherein the labeled carrier protein domain detects the POI.

In a detailed embodiment the microarray further comprises individual units of enzymes derived from libraries of enzymes, coenzymes derived from libraries of coenzymes and synthetic appendage labels derived from libraries of synthetic appendage labels, wherein the individual units of enzymes, coenzymes and synthetic appendage labels are spatially addressed on a three dimensional object. In a detailed aspect the POI is a biosynthetic enzyme. In a further detailed aspect, the biosynthetic enzyme is selected from a polyketide (PK) synthase, a non-ribosomal peptide (NRP) synthase, or a fatty acid (FA) synthase. In a further detailed aspect, the polyketide (PK) synthase comprises at least one domain with acyl carrier protein (ACP) activity. In a further detailed aspect, the non-ribosomal peptide (NRP) synthase comprises at least one domain with peptidyl carrier protein (PCP), aryl carrier protein (ArCP) and/or acyl carrier protein (ACP) activity. In a further detailed aspect, the fatty acid (FA) synthase comprises at least one domain with acyl carrier protein (ACP) and/or aryl carrier protein (ArCP) activity. In a further detailed aspect, the biosynthetic enzyme comprises a hybrid between a FA synthase, PK synthase, and/or NRP synthase and further comprises at least one domain with acyl carrier protein (ACP) and/or aryl carrier protein (ArCP) activity. In a further detailed aspect, the carrier protein-enzyme-coenzyme complex further comprises coenzyme A (CoA) or a derivative thereof. In a further detailed aspect, the carrier protein-POI-coenzyme complex further comprises a phosphotransferase enzyme. In a further detailed aspect, the phosphotransferase enzyme is a 4′-phosphopantetheinyl transferase.

In a further detailed aspect, the synthetic appendage label further comprises a linker and a reporter. In a further detailed aspect, the reporter is an affinity reporter, a colored reporter, a fluorescent reporter, a magnetic reporter, a radioisotopic reporter, a peptide label, a metal reporter, a nucleic acid reporter, a lipid reporter, a glycosylation reporter, or a reactive reporter. In a further detailed aspect, the reporter is a precursor to an affinity reporter, a colored reporter, a fluorescent reporter, a magnetic reporter, a radioisotopic reporter, a peptide reporter, a metal reporter, a nucleic acid reporter, a lipid reporter, a glycosylation reporter, or a reactive reporter. In a detailed embodiment, the microarray further comprises interaction with a secondary molecule to detect or modulate a function of the label. In a further aspect, the secondary molecule is selected from a carbohydrate, a protein, a peptide, an oligonucleotide, or a synthetic receptor.

In a detailed embodiment, the microarray further comprises a profiler to measure individual responses of the biosynthetic enzyme to given conditions to identify the biosynthetic enzyme. In a detailed embodiment, the microarray further comprises a product generated from the transfer of the synthetic appendage label to the carrier protein is removed chemically or enzymatic ally.

### BRIEF DESCRIPTION OF THE DRAWINGS

FIG. 1 shows routes to synthesis of modified derivatives of CoA.

FIG. 2 shows modified derivatives of CoA containing fluorescent and/or colored synthetic appendage labels or an affinity-based synthetic appendage label.

FIG. 3 shows the post-translational 4′-phosphopantetheinylation of carrier protein domains and shows the modified addition of coenzyme A analogs onto conserved serine residues within apo-carrier protein domains.

FIG. 4 shows an application of the composition and method to identify proteins that contain a Type I fatty acid ACP.

FIG. 5 shows an application of the composition and method to identify proteins that contain a Type II fatty acid ACP.

FIG. 6 shows an application of the composition and method to identify proteins within modular Type I PK synthases. The compositions and methods identify DEBS1, a synthase involved in the biosynthesis of erythromycin.

FIG. 7 shows an application of the composition and method to identify proteins within iterative Type I PK synthases. The compositions and methods identify 6MSAS, a protein responsible for the biosynthesis of 6-methylsalicylic acid.

FIG. 8 shows an application of the composition and method to identify proteins within Type II PK synthases. The compositions and methods identify a carrier protein domain used in the biosynthesis of actinorhodin.

FIG. 9 shows an application of the composition and method to identify proteins within NRP synthases. The compositions and methods identify a carrier protein domain used in the biosynthesis of tyrocidine.

FIG. 10 shows an application of the composition and method to tag fusion molecules with an SAFP-TAG.

FIG. 11 shows the use of this method to identify recombinant VibB within the cell lysate of a recombinant organism (E. coli). FIG. 11A shows the structure of the synthase screened and, FIG. 11B depicts the effects of different fluorescent reporter groups on identifying VibB in crude lysate. FIG. 11C shows the effects of different fluorescent reporter groups on the labeling of purified VibB. Lanes A-C are denoted by A=BODIPY FL, B=N-7-dimethylamino-4-methylcoumarin and C=Oregon Green 488.).

FIG. 12 shows the affinity recognition of proteins containing native and engineered carrier protein domains. (A) Structure of biotin-containing CoA analog used, (B) Western blot analysis illustrates the use of affinity recognition to indentify natively expressed proteins EntB and EntF in E. coli lysate, (C) Western blot analysis illustrates affinity recognition technique selects recombinant proteins (VibB in E. coli) via blot analysis. Lanes 1-9 denote a decreasing concentration of the of biotin-containing CoA analog.

FIG. 13 shows affinity purification of VibB. (A) Structure of biotin-containing CoA analog used, (B) Western blot analysis illustrates the used of affinity recognition to purify recominant VibB from E. coli lysate,

FIG. 14 shows proteolytic digestion of a synthase to identify the relative uptake of a fluorescent or affinity reporter within crypto-modified carrier protein domains.

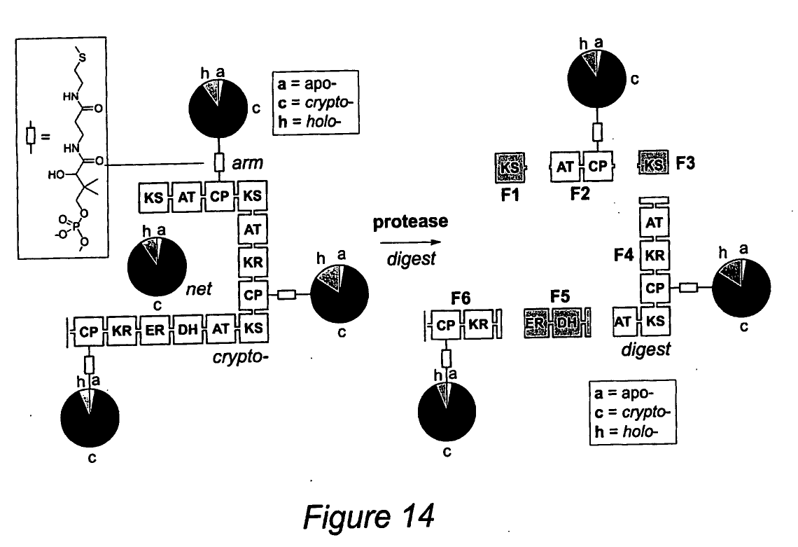

FIG. 15 shows radioactive uptake into the products and product intermediates of synthases partially blocked by crypto-modification.

FIG. 16 shows radioactive uptake into proteolytic fragments of synthases containing carrier protein domains.

FIG. 17 shows a system for combinatorial screening of carrier protein (CP) domains.

FIG. 18 shows a carrier protein profiler.

FIG. 19 shows functional manipulation of carrier proteins by fluorescent visualization.

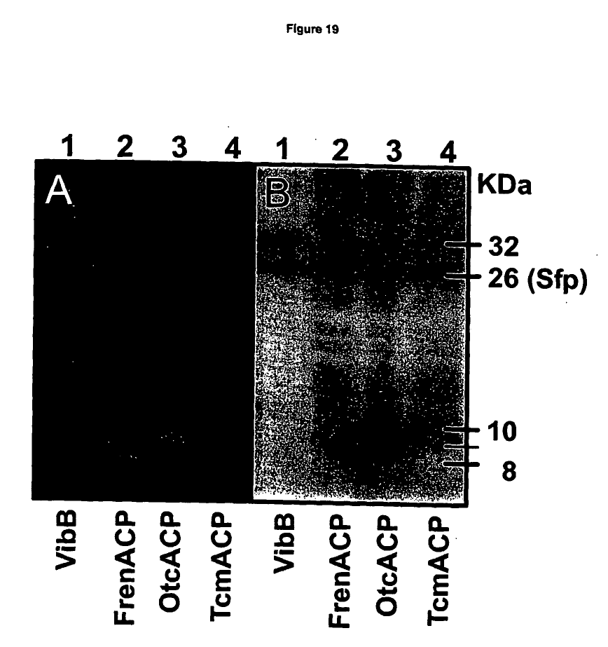

FIG. 20 shows relative Sfp activity in engineered systems.

FIG. 21 shows a Western blot analysis of a natural product synthase from a natural producer, 6-deoxyerythronolide B synthase from Saccharopolyspora erythraea.

### DETAILED DESCRIPTION OF ILLUSTRATIVE EMBODIMENTS

“Biosynthetic enzymes” refers to enzymes involved in secondary metabolic biosynthesis. Non-ribosomal peptide (NRP) synthase, polyketide (PK) synthase, fatty acid synthase are examples of biosynthetic enzymes. Biosynthetic enzymes are useful for secondary metabolic biosynthetic pathways, for example, non-ribosomal peptide, polyketide, carbohydrate, terpene, sterol, shikimic acid, and fatty acid pathways

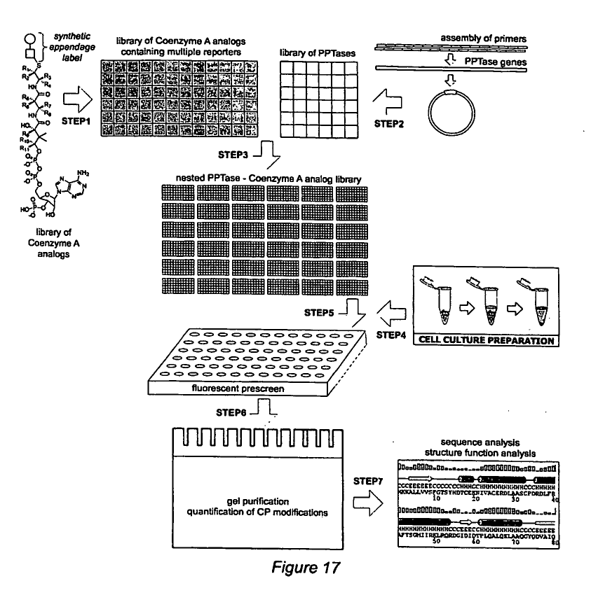

“Coenzyme” refers to a catalytically active, low molecular mass component of an enzyme; and also refers to a dissociable, low-molecular mass active group of an enzyme that transfers chemical groups or hydrogen or electrons. Coenzyme A (CoA) is an exemplary coenzyme. Non-natural coenzyme derivatives, for example, non-natural coenzyme A derivatives, can be synthesized to contain derivatives of the natural CoA molecule with variant moieties at key locations on the molecule. For instance, a library of derivatized functionality at backbone carbons within the pantothenate, beta-alanine, and cystamine sub-groups of pantetheine can be created. These derivatives can contain variation within the functionality within the pantetheine backbone as given by R₁-R₁₁ as shown in FIG. 17. Modifications about R₁-R₁₁ can include the appendage of alkyl, alkoxy, aryl, aryloxy, hydroxy, halo, and/or thiol groups.

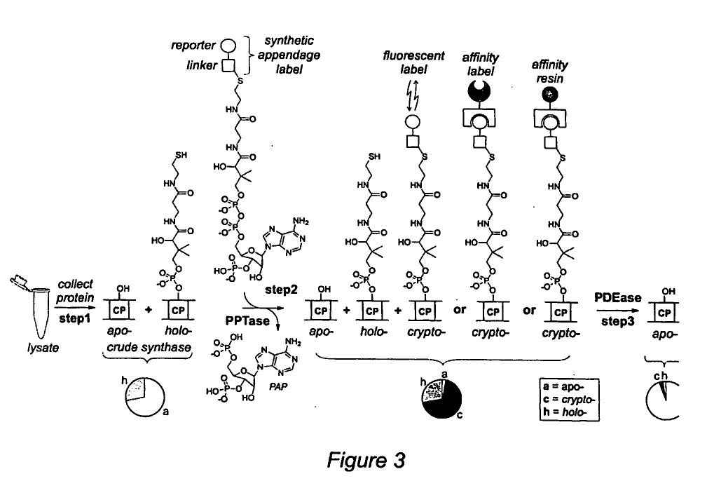

“Synthetic appendage label” refers to a detectable label attached to the coenzyme molecule that is transferred to the carrier protein domain of the biosynthetic enzyme to label the biosynthetic enzyme. This label consists of a linker and reporter (FIG. 3), wherein the linker serves to attach to the thiol of the coenzyme and the reporter provides a signal for analytical processing. An affinity reporter can serve to isolate and purify the biosynthetic enzyme. Derivation or modification can appear within the choice of reporter or tag. Derivation or modification can include the appendage of different dyes, affinity reporters and/or linkers. These modifications can include multimeric derivatives, including but not limited to, functional groups that contain more than one fluorescent or affinity reporter and/or a combination of fluorescent and affinity reporters. Ideally each member of the library should either contain a fluorescent reporter or express an affinity that can bind to a material containing a fluorescent reporter.

“Carrier protein domain” refers to a domain within the biosynthetic enzyme. The carrier protein domain can be labeled with the synthetic appendage label that is catalytically transferred from the coenzyme, for example, coenzyme A.

“apo-synthase” or “apo-carrier protein” refers to a synthase containing a carrier protein, a carrier protein or a peptide portion of a carrier protein that contains a serine residue that can be 4′-phosphopantetheinylated, but is not 4′-phosphopantetheinylated. The term “apo-” denotes a state of protein modification.

“holo-synthase” or “holo-carrier protein” refers to a synthase containing a carrier protein, a carrier protein or a peptide portion of a carrier protein that contains a serine residue that has been 4′-phosphopantetheinylated by natural Coenzyme A. The term “holo-” denotes a state of protein modification.

“crypto-synthase” or “crypto-carrier protein” refers to a synthase containing a carrier protein, a carrier protein or a peptide portion of a carrier protein that contains a serine residue that has been 4′-phosphopantetheinylated by a modified derivative of Coenzyme A bearing a synthetic appendage label. The term “crypto-” denotes a state of protein modification.

“Carrier protein-enzyme-coenzyme complex” refers to derivatives of coenzyme A labeled with a synthetic appendage label that transfer the label and selectively mark an acyl carrier protein domain. The acyl carrier protein domain is a domain within the biosynthetic enzyme. The attachment of the label provides a device for selection, identification and/or recognition of the biosynthetic enzyme. This process arises through the formation of an enzyme-coenzyme complex. Formation of this complex can occur prior to or after the formation of a complex between the enzyme and its carrier protein substrate. The enzyme-coenzyme complex and/or carrier protein-enzyme-coenzyme complex is modified by the appendage of a label.

“Array” or “microarray” refer to various techniques and technologies that can be used for synthesizing dense arrays of biological materials on or in a substrate or support. For example, microarrays are synthesized in accordance with techniques sometimes referred to as VLSIPS™ (Very Large Scale Immobilized Polymer Synthesis) technologies. Some aspects of VLSIPS™ and other microarray and polymer (including protein) array manufacturing methods and techniques have been described in U.S. Ser. No. 09/536,841, WO 00/58516, U.S. Pat. Nos. 5,143,854, 5,242,974, 5,252,743, 5,324,633, 5,445,934, 5,744,305, 5,384,261, 5,405,783, 5,424,186, 5,451,683, 5,482,867, 5,491,074, 5,527,681, 5,550,215, 5,571,639, 5,578,832, 5,593,839, 5,599,695, 5,624,711, 5,631,734, 5,795,716, 5,831,070, 5,837,832, 5,856,101, 5,858,659, 5,936,324, 5,968,740, 5,974,164, 5,981,185, 5,981,956, 6,025,601, 6,033,860, 6,040,193, 6,090,555, 6,136,269, 6,269,846, 6,022,963, 6,083,697, 6,291,183, 6,309,831 and 6,428,752, in PCT Applications Nos. PCT/US99/00730 (International Publication Number WO 99/36760) and PCT/US01/04285, which are all incorporated herein by reference in their entireties for all purposes. Patents that describe synthesis techniques in specific embodiments include U.S. Pat. Nos. 5,412,087, 6,147,205, 6,262,216, 6,310,189, 5,889,165, and 5,959,098, hereby incorporated by reference in their entireties for all purposes. Nucleic acid arrays are described in many of the above patents, but the same techniques may be applied to polypeptide arrays.

“Array” or “microarray” further refer to a collection of molecules that can be prepared either synthetically or biosynthetically. The molecules in the array may be identical, they may be duplicative, and/or they may be different from each other. The array may assume a variety of formats, e.g., libraries of soluble molecules; libraries of compounds tethered to resin beads, silica chips, or other solid supports; and other formats.

“Solid support,” “support,” or “substrate” refer to a material or group of materials having a rigid or semi-rigid surface or surfaces. In many embodiments, at least one surface of the solid support will be substantially flat, although in some embodiments it may be desirable to physically separate synthesis regions for different compounds with, for example, wells, raised regions, pins, etched trenches, or other separation members or elements. In some embodiments, the solid support(s) may take the form of beads, resins, gels, microspheres, or other materials and/or geometric configurations.

“Probe” refers to a molecule that can be recognized by a particular target. To ensure proper interpretation of the term “probe” as used herein, it is noted that contradictory conventions exist in the relevant literature. The word “probe” is used in some contexts to refer not to the biological material that is synthesized on a substrate or deposited on a slide, as described above, but to what is referred to herein as the “target.” A target is a molecule that has an affinity for a given probe. Targets may be naturally occurring or man-made molecules. Also, they can be employed in their unaltered state or as aggregates with other species. The samples or targets are processed so that, typically, they are spatially associated with certain probes in the probe array. For example, one or more tagged targets may be distributed over the probe array.

Targets can be attached, covalently or noncovalently, to a binding member, either directly or via a specific binding substance. Examples of targets that can be employed in accordance with this invention include, but are not restricted to, antibodies, cell membrane receptors, monoclonal antibodies and antisera reactive with specific antigenic determinants (such as on viruses, cells or other materials), drugs, oligonucleotides, nucleic acids, peptides, cofactors, lectins, sugars, polysaccharides, cells, cellular membranes, and organelles. Targets are sometimes referred to in the art as anti-probes. As the term target is used herein, no difference in meaning is intended. Typically, a “probe-target pair” is formed when two macromolecules have combined through molecular recognition to form a complex.

“Microarray” refers to libraries of compounds immobilized on a surface of a solid support wherein each individual unit of compound is localized in a predetermined region of the solid support. The addressing of individual units of compounds allows interaction with a complex mixture to identify components within the complex mixture. For example, libraries of coenzymes and synthetic appendage labels immobilized on a surface of a solid support, wherein each individual unit of coenzyme or synthetic appendage label is localized in a predetermined region of the solid support surface; allowing interaction of carrier protein domains of the biosynthetic enzyme, coenzyme and synthetic appendage label to uniquely identify a biosynthetic enzyme, wherein the biosynthetic enzyme is within a solution, complex mixture or cell culture.

“Spatially addressed on a three dimensional object” refers to libraries of coenzyme or synthetic appendage label localized to a predetermined region of a solid support surface, for example, as a microarray.

“Library” refers to a collection of individual units of coenzymes or synthetic appendage labels with affinity for carrier protein domains within biosynthetic enzymes. Specificity of individual units of coenzymes and synthetic appendage labels for carrier protein domains within biosynthetic enzymes allows identification of specific biosynthetic enzymes within a solution, complex mixture or cell extract.

### Exemplary Embodiments

### EXAMPLE 1

Biosynthesis of Natural Products Derived from Fatty Acid (FA), Polyketide (PK) and Non-Ribosomal Peptide (NRP)

A common theme in the biosynthesis of FAs, PKs, NRPs, in a producer organisms is the post-translational modification of their synthases by 4′-phosphopantetheinyltransferase (PPTase) See FIG. 3. Specifically, the carrier proteins of each biosynthetic enzyme system is modified with a 4′-phosphopantetheine moiety derived from coenzyme A (CoA) at a conserved serine residue. In all instances, this modification, from the apo-carrier protein to the 4′-phosphopantetheinylated holo-carrier protein, is essential for biosynthesis for each class these small molecules. Of all bacterial PPTases, Sfp, responsible for modifying surfactin synthase in Bacillus subtilis, is commonly used to modify PK and NRP synthases for in vitro and in vivo studies because it demonstrates the broadest activity of all known PPTases implicated in secondary metabolite biosyntheses. An interesting characteristic of Sfp is its ability to accept functionalized CoA thioesters as substrates. This ability has been utilized to transfer pre-loaded 4′-phosphopantetheine moieties onto carrier protein domains in order to study non-natural amino acid or ketide substrates.

The post-translational 4′-phosphopantetheinylation of carrier protein domains in natural systems is shown in step 1 of FIG. 3. A 4′-phosphopantetheinyl transferase (PPTase) serves to transfer 4′-phosphopantetheine from coenzyme A to a conserved serine within the carrier protein as given by the natural conversion of apo-carrier protein to holo-carrier protein. This process arises through the formation of an enzyme-coenzyme complex. Formation of this complex can occur prior to or after the formation of a complex between the enzyme and its carrier protein substrate. This process results in the production of a 4′-phosphopantetheinylated carrier protein and 3′,5′-adenosine bisphosphate (PAP). PAP can be further modified by a phosphatase or nucleotidase. This can include conversion to AMP. 4′-Phosphopantetheinylated carrier protein domains can be dephosphopantetheinylated by the action of a phosphodiesterase such as acyl-carrier-protein phosphodiesterase (ACP-PDE). Characterization of this phosphodiesterase activity has not yet been identified in natural PK and/or NRP systems.

The post-translational 4′-phosphopantetheinylation of carrier protein domains in modified system is shown in Step 2 of FIG. 3. A modified system was engineered to incorporate a recognizable synthetic appendage label during the 4′-phosphopantetheinylation reaction. Here derivatives of coenzyme A selectively mark an acyl carrier protein domain with a synthetic appendage label containing a reporter. This reporter is depicted by a sphere. The attachment of this label provides a device to for selection, identification and/or recognition. This process arises through the formation of an enzyme-coenzyme complex. Formation of this complex can occur prior to or after the formation of a complex between the enzyme and its carrier protein substrate. The enzyme-coenzyme complex and/or enzyme-coenzyme-substrate complex is modified by the appendage of a label. This process results in the production of 4′-phosphopantetheinylated carrier protein and 3′,5′-adenosine bisphosphate (PAP). PAP can be further modified by a phosphatase or nucleotidase. This can include conversion to AMP. 4′-Phosphopantetheinylated carrier protein domains can be dephosphopantetheinylated by the action of an phosphodiesterase such as acyl-carrier-protein phosphodiesterase (ACP-PDE). Characterization of this phosphodiesterase activity has been identified in the reversal of PK and/or NRP systems.

Additional modification can arise through the addition of phosphatases. In particular, nucleotidases such as 3′(2′),5′-bisphosphate nucleotidase (E.C.3.1.3.7) can be used to convert PAP to adenosine 5′-phosphate (AMP) as shown in FIG. 3. This process serves to inhibit the reversal of a given 4′-phosphopantetheinylation step. Phosphodiesterases such as an acyl-carrier-protein phosphodiesterase (ACP-PDE) or EC 3.1.4.14 can be used to convert the modified 4′-phosphopantetheinylated carrier protein back to its native state. This ACP-PDE serves to convert crypto-carrier proteins back to its apo-state, therein providing native materials for biochemical study.

Having identified PPTase activity to be a unifying marker of FA, NRP and PK biosynthesis, the question is whether Sfp could transfer modified CoA derivatives other than thioester-linked substrates. FIG. 3 illustrates the utility of modified CoA derivatives for tagging carrier protein mediated biosynthetic enzymes. The following section describes a series of FA, PK and NRP systems applicable to this method.

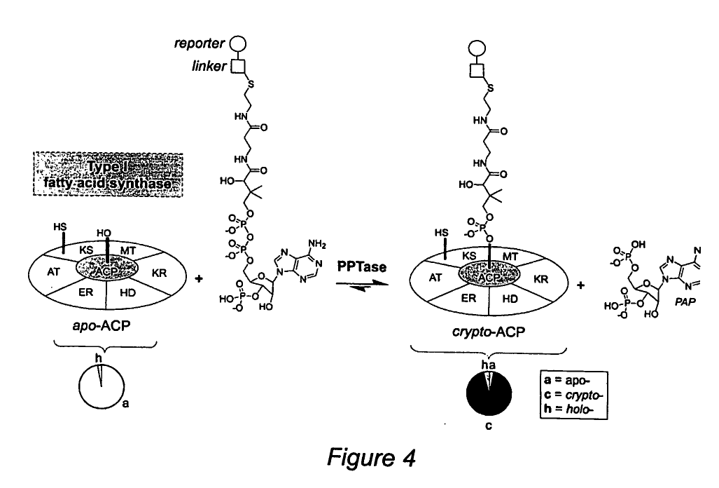

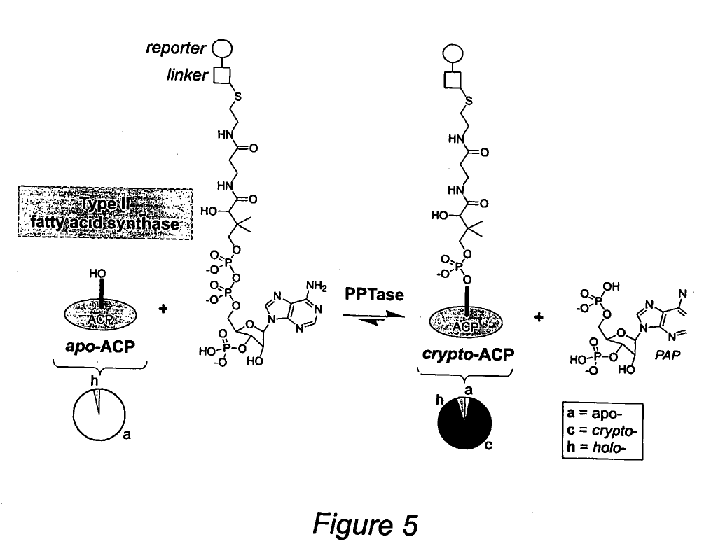

An application of the method to identify proteins that contain a fatty acid ACP is shown in FIGS. 4 and 5. The examples shown here illustrate the use of modified CoA derivatives to identify ACP domains within Type I and Type II FA synthases. This process results in the production of 3′,5′-adenosine bisphosphatate (PAP). Decomposition of PAP through the action of nucleotidases serves as a mechanism to inhibit reversibility of the labeling reaction. The products of this reaction (right) can be processed with a phosphodiesterase or an acyl-carrier-protein phosphodiesterase (ACP-PDE). This phosphodiesterase serves to convert the ACP to its native form.

Fatty acid synthetases (FASs) are categorized as either Type I or Type II depending upon their protein structure (FIGS. 4 and 5). Prokaryotes produce Type II FASs in which all domains (ACP=acyl carrier protein, KS=beta-ketoacyl ACP synthase, AT=acetyl CoA ACP transacetylase, MT=malonyl CoA ACP transferase, KR=beta-ketoacyl ACP reductase, HD=beta-hydroxyacyl ACP dehydratase, and ER=enoyl ACP reductase) exist as independent proteins. These proteins then converge to a multimeric complex, presumably with holo-ACP located at the center and the other enzymes encircling the ACP. Eukaryotes produce Type I FASs in which the domains exist as either one or two polypeptide chains, with one domain located behind the other in protein and gene sequence. In both Type I and Type II FASs, the ACP must be converted from apo-ACP to holo-ACP through post-translational activity of a PPTase, which transfers 4′-phosphopantetheine from CoA to a conserved serine in the ACP. PPTase activity is demonstrated on both Type I and Type II ACPs to transfer modified CoA, thereby incorporating a modification in the crypto-ACP through transfer of a modified 4′-phosphopantetheine of a derivatized CoA.

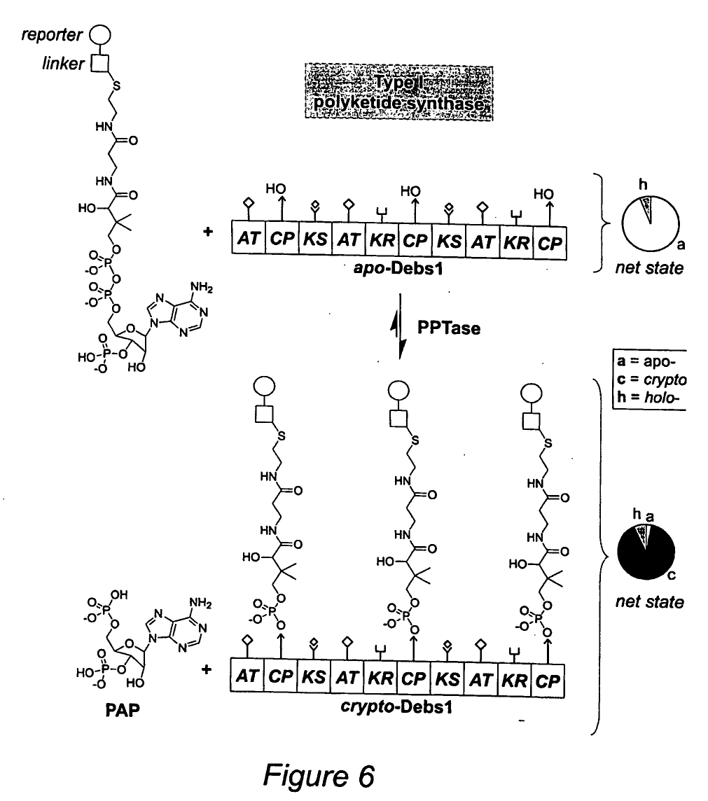

An application of the method to identify proteins within modular Type I PK synthases is shown in FIG. 6. This example illustrates the use of this system to identify DEBS1, a synthase involved in the biosynthesis of erythromycin. A PPTase serves to 4′-phosphopantetheinylate up to 3 ACPs within the DEBS1 protein. The DEBS1 protein is then recognized the covalent attachment of a synthetic appendage label containing a linker (box) and reporter (sphere). Only one of the three ACP domains within the DEBS1 protein must be tagged with a label to be identified. This process results in the production of 3′,5′-adenosine bisphosphatate (PAP). Decomposition of PAP through the action of nucleotidases serves as a mechanism to inhibit reversibility of the labeling reaction. The products of this reaction (below) can be processed with a phosphodiesterase or an acyl-carrier-protein phosphodiesterase (ACP-PDE). This phosphodiesterase serves to convert the ACP to its native form.

DEBS1 is the first module in the biosynthesis of 6-deoxyerythronolide B, the precursor to the antibiotic erythromycin produced by Saccharopolyspora erythraea (FIG. 6). DEBS1, a prototypical Type I polyketide synthase (ACP=acyl carrier protein, KS=beta-ketoacyl ACP synthase, AT=acetyl CoA ACP transacetylase, KR=beta-ketoacyl ACP reductase, DH=beta-hydroxyacyl ACP dehydratase, and ER=enoyl ACP reductase). DEBS1 contains three ACP domains, three AT domains, two KS domains, and two KR domains. Apo-DEBS1 protein is first translated from the mRNA, followed by post-translational activity of a PPTase, which transfers 4′-phosphopantetheine from CoA to a conserved serine in each ACP. PPTase activity is demonstrated by transferring a modified CoA, thereby incorporating a modification into each crypto-ACP through transfer of a modified 4′-phosphopantetheine of a derivatized CoA. DEBS1 can incorporate three modifications, one for each ACP domain found in the protein.

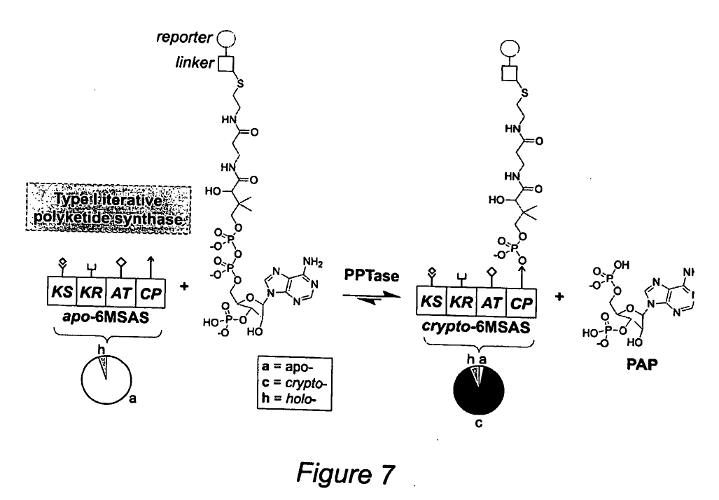

An application of the method to identify proteins within iterative Type I PK synthases is shown in FIG. 7. This example illustrates the use of this system to identify 6MSAS, a protein responsible for the biosynthesis of 6-methylsalicylic acid. A PPTase serves to 4′-phosphopantetheinylate a single ACP within 6MSAS. The 6MSAS protein is then recognized the covalent attachment of a synthetic appendage label containing a linker (box) and reporter (sphere). This process results in the production of 3′,5′-adenosine bisphosphatate (PAP). Decomposition of PAP through the action of nucleotidases serves as a mechanism to inhibit reversibility of the labeling reaction. The products of this reaction (crypto-6MSAS) can be processed with a phosphodiesterase or an acyl-carrier-protein phosphodiesterase (ACP-PDE). This phosphodiesterase serves to convert the ACP to its native form.

6MSAS is the enzyme involved in the biosynthesis of 6-methyl salicylic acid produced by Penicillium patulum (P. griseofulvum) As illustrated in FIG. 7,this iterative Type I polyketide synthetase contains one ACP domain, one KS domain, one AT domain, and one KR domain. Apo-6MSAS protein is translated from mRNA whereby post-translational activity of a PPTase transfers 4′-phosphopantetheine from CoA to a conserved serine in each ACP. PPTase activity accepting a modified CoA is demonstrated, thereby incorporating a modification into the crypto-ACP through transfer of a modified 4′-phosphopantetheine of a derivatized CoA. 6MSAS can incorporate one modification at the ACP domain.

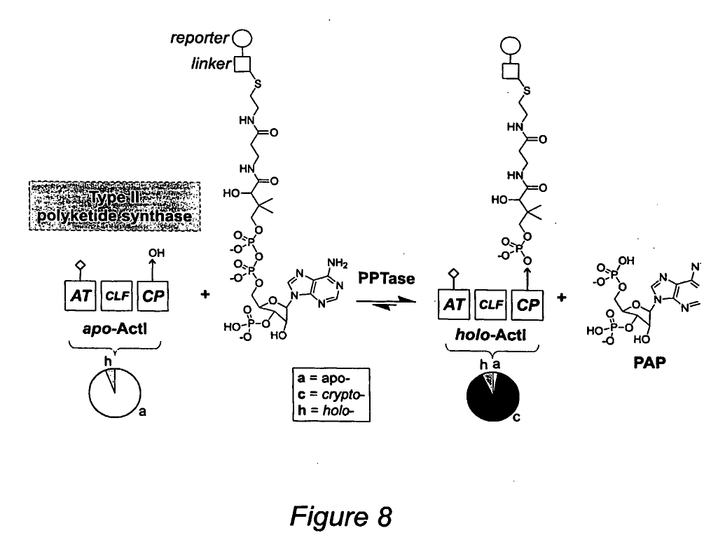

An application of the method to identify proteins within Type II PK synthases is shown in FIG. 8. This example illustrates the use of this system to identify the carrier protein domain used in the biosynthesis of actinorhodin. A PPTase serves to 4′-phosphopantetheinylate a single standalone ACP. This standalone ACP is then recognized the covalent attachment of a synthetic appendage label containing a linker (box) and reporter (sphere). This process results in the production of 3′,5′-adenosine bisphosphatate (PAP). Decomposition of PAP through the action of nucleotidases serves as a mechanism to inhibit reversibility of the labeling reaction. The products of this reaction (crypto-state) can be processed with a phosphodiesterase or an acyl-carrier-protein phosphodiesterase (ACP-PDE). This phosphodiesterase serves to convert the ACP to its apo-form.

The ActI genes from Streptomyces coelicolor actinorhodin biosynthesis contain what is referred to as a minimal Type II PK synthase, which consists of the ketosynthase (KS), chain-length factor (CLF), and an acyl carrier protein (ACP) (FIG. 8). The ActI genes come from Streptomyces coelicolor and represent the prototypical minimal PK synthase of the Type II variety. Post-translational modification of the ActI apo-ACP is performed by a PPTase, which transfers 4′-phosphopantetheine from CoA to a conserved serine in each ACP. PPTase activity transferring a modified CoA is demonstrated, thereby incorporating a modification into the crypto-ACP through transfer of a modified 4′-phosphopantetheine of a derivatized CoA. The ActI ACP contains one modification at the ACP domain.

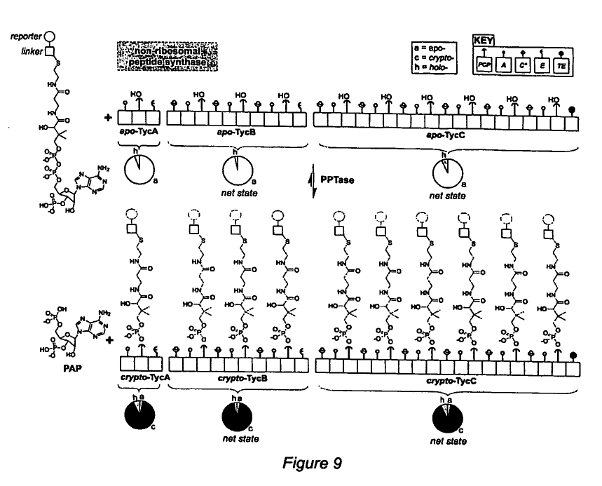

An application of the method to identify proteins within NRP synthases is shown in FIG. 9. This example illustrates the use of this system to identify the carrier protein domain used in the biosynthesis of Tyrocidine. A PPTase serves to 4′-phosphopantetheinylate peptidyl carrier protein domains (PCP) within TycA, TycB, and TycC. TycA contains one PCP, while TycB contains multiple PCP domains. TycB and TycC require the labeling of at least one of their PCP modules to be identified by this method. This process results in the production of 3′,5′-adenosine bisphosphatate (PAP). Decomposition of PAP through the action of nucleotidases serves as a mechanism to inhibit reversibility of the labeling reaction. The products of this reaction (crypto-states) can be further processed with a phosphodiesterase or an acyl-carrier-protein phosphodiesterase (ACP-PDE).

Tyrocidine C, a cyclic decapeptide topical antibiotic produced by Bacillus brevis, is biosynthesized through the activity of three enzymes, TycA, TycB, and TycC NRP synthases (FIG. 9). TycA contains one module (loads one amino acid) with one A (adenylation) domain, one PCP (peptidyl carrier protein) domain, and one E (epimerization) domain. TycB contains three modules (loads three amino acids) and contains three A domains, three PCP domains, three C (condensation) domains, and one E domain. TycC contains six modules (loads six amino acids) and contains six A domains, six PCP domains, six C domains and one TE (thioesterase) domain. Post-translational modification of all ten apo-PCPs in TycA, B, and C is performed by a PPTase, which transfers 4′-phosphopantetheine from CoA to a conserved serine in each carrier protein. PPTase activity transferring a modified CoA is demonstrated, thereby incorporating a modification into the crypto-PCP through transfer of a modified 4′-phosphopantetheine from a derivatized CoA. Each carrier protein domain in TycA, TycB and TycC can incorporate one modification per domain.

### EXAMPLE 2

Preparation of Modified CoA Derivatives

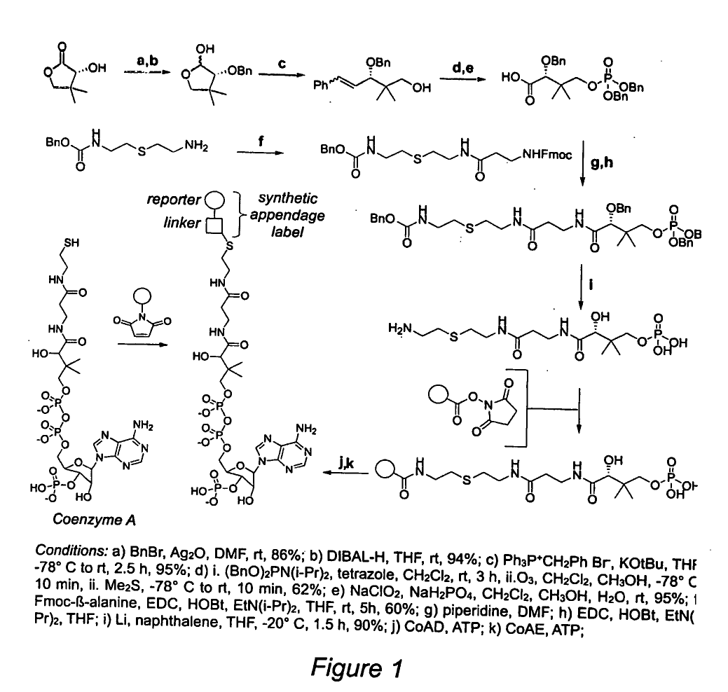

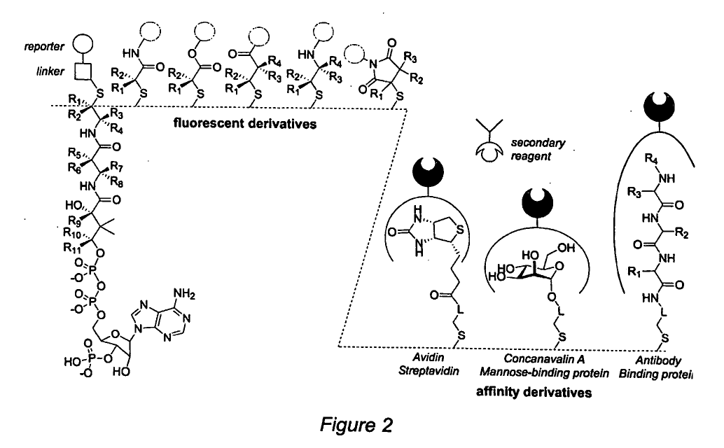

Coenzyme A (CoA) can be selectively tagged with a synthetic appendage label at the free thiol through reactivity with soft electrophiles such as enones (i.e., α,β-unsaturated ketones or maleimides), α-haloketones, α-haloesters, and/or α-haloamides (FIG. 1). These synthetic appendage labels can include, but not limited to, fluorescent or colored dyes and/or affinity reporters (FIG. 2), such as biotin, mannose or other carbohydrates, oligopeptides, or oligo nucleotides. These reporters are covalently attached to the soft electrophile through a flexible or rigid linker. Therefore, incubating CoA with a soft electrophile-linked marker results in the covalent attachment of the marker onto the CoA (crypto-state, FIG. 3). The CoA-synthetic appendage entity may also be synthesized de novo using chemical or chemo-enzymatic methods (FIG. 1).

The fluorescent and/or colored derivatives of CoA are depicted in FIG. 2. An illustration of the fluorescent analogs wherein the sphere represents a reporter unit and the box represents a linker. Structures of a selection of derivatives wherein R₁-Rₙ represent functionality that includes but is not limited to alkyl, aryl, alkoxy, aryloxy, halo, sulfoxy, sulfonyl, ester, and/or nitrile groups. The reporter D can be but is not limited to Alexa Fluor Derivatives, BODIPY Derivatives, Fluorescein Derivatives, Oregon Green Derivatives, Eosin Derivatives, Rhodamine Derivatives, Texas Red Derivatives, Pyridyloxazole Derivatives, Benzoxadiazole Derivatives, NBD derivatives, SBD (7-fluorobenz-2-oxa-1,3-diazole-4-sulfonamide), IANBD derivatives, Lucifer Yellow derivatives, Cascade Blue dye, Cascade Yellow dye, Dansyl derivatives, Dapoxyl derivatives, Dialkylaminocoumarin derivatives, Eosin, Erythrosin, Hydroxycoumarin derivatives, Marina Blue dye, Methoxycoumarin derivatives, Pacific Blue dye. These dyes are attached through linker L.

The affinity-based derivatives of CoA are depicted in FIG. 2. (A) An illustration of the affinity analogs wherein the sphere represents an affinity-based reporter. Recognition of this reporter is possible through the action of a biomolecule and a secondary reagent. Structures of a selection of derivatives that contain a series of tags, including but not limited to the use of a biotin, carbohydrate, or peptide tags. Biotinylated derivatives can be selected by its high affinity binding to Avidin and/or Streptavidin, and fusion proteins developed thereon. The detection of biotin-labeled CP can be accomplished using fusion proteins developed from Streptavidin and/or avidin. Carbohydrate derivatives can be identified by their binding to carbohydrate-binding proteins. The example shown illustrates the recognition of a β-mannopyranoside by Concanavalin A. Peptide-tags can be recognize either by metals, metal ions, proteases, peptide binding proteins and/or antibodies. The example shown illustrates the recognition of a peptide tag. Peptide tags can be made from peptides with a variety of functionality (R₁-Rₙ) and length.

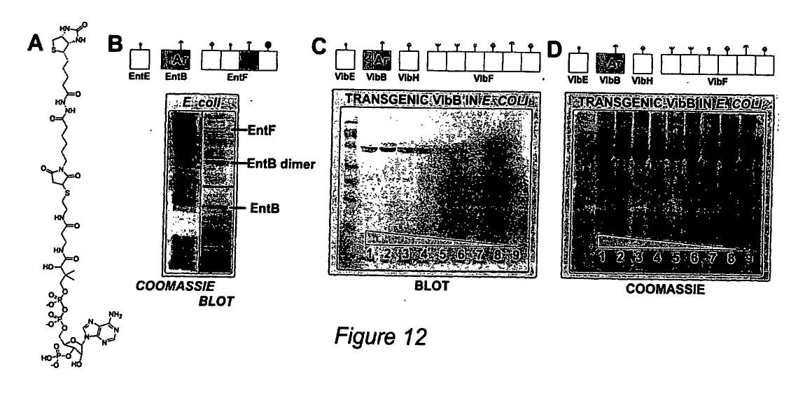

Exemplary experimental procedure: Coenzyme A disodium salt (300 ug, 0.37 umol) in 1.9 mL MES acetate 100 mM Mg(OAc)2 buffer, pH 6.0, is diluted with 300 uL DMSO, and mixed with a thiol reactive tag pre-dissolved in DMSO (as given by BODIPY=4.8 uL of 25 mg/mL solution of BODIPY® FL N-(2-aminoethyl)maleimide (Molecular Probes, Seattle, Wash.), DACM=13.5 uL of a 10 mg/mL solution of N-(7-dimethylamino-4-methylcoumarin-3-yl)maleimide (Molecular Probes, Seattle, Wash.), OG=a 8.7 uL of a 10 mg/mL solution of Oregon Green® 488 maleimide (Molecular Probes, Seattle, Wash.), or BIOTIN-B1=5.2 uL of a 25 mg/mL solution of biotin B1 as shown in FIG. 12 (Quanta Biodesign, Powell, Ohio). The solution is vortexed briefly, cooled for 30 min at 0° C., incubated at room temp for 10 min, and washed with ethyl acetate (3 times with 10 mL). Alternatively, the excess tag can be removed by surfaces, beads or gels containing terminal thiols.

### EXAMPLE 3

Tagging Heterologously Expressed Carrier Protein Domains

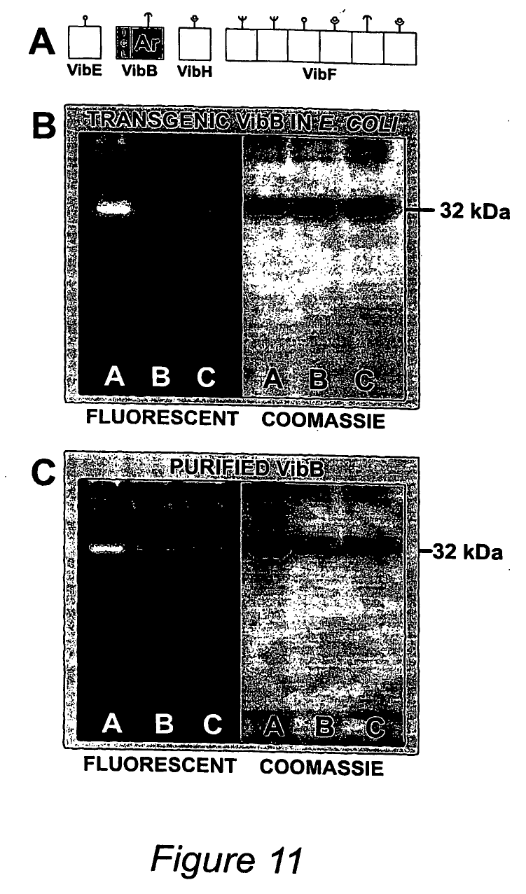

Fluorescent tagging with derivatives in FIG. 2 was repetitively conducted on proteins from crude cell lysate from recombinant E. coli BL21 cells expressing a carrier protein (i.e., VibB). Cell lysate was dialyzed to remove small molecules (<3 or <10 kDa), incubated with CoA-DYE and recombinant Sfp, and analyzed by SDS-PAGE. The outcome of this experiment is provided in FIG. 11. When viewed under irradiation, recombinant VibB is visualized as a fluorescent band that was verified with two methods. First, standard Coomasie staining showed the fluorescent band to have the proper molecular weight when compared to molecular weight markers. Second, an identical gel was electrophoretically transferred to a polyvinylidene fluoride (PVDF) membrane, and the fluorescent band was excised from the membrane. This membrane piece was subjected to N-terminal amino acid sequencing by Edman degradation. The first 10 amino acids of the returned sequence, MAIPKIASYP, mapped to the correct protein, VibB, when searched with BLAST against 1.4 million sequences in GenBank. Broad applicability of these techniques is anticipated for validating proper folding and modification ability of recombinant PK and NRP systems.

One liter of E. coli BL21 (de3) cells, grown using standard methods of IPTG induced overexpression of recombinant proteins, were lysed by sonication at 0° C. in 30 ml of 0.1M Tris-Cl pH 8.0 with 1% glycerol in the presence of 500 uL of a 10 mM phenylmethanesulfonyl fluoride (PMSF) solution in isopropanol with 50 uL of a protease inhibitor cocktail (A mixture of protease inhibitors with broad specificity for the inhibition of serine, cysteine, aspartic and metallo-proteases, and aminopeptidases. Contains 4-(2-aminoethyl)benzenesulfonyl fluoride (AEBSF), pepstatin A, E-64, bestatin, and sodium EDTA, Sigma-Aldrich Inc.). After centrifugation at 4000×g for 10 min, 200 uL of this cell lysate is treated with 80 uL of the dye-CoA solution (see Preparation of modified CoA derivatives) and 1 uL (30 ug) of 30 mg/mL purified Sfp, and the reaction is incubated at room temperature for 30 min in darkness. A 800 uL aliquot of a 10% trichloroacetic acid solution is added and cooled at −20° C. for 30-60 min. The samples are centrifuged at 14000×g for 4 minutes, and the supernatant is removed. The pellets are resuspended in 1:1 mixture of 1.0 M Tris-HCl pH 6.8 and 2× SDS-PAGE sample buffer (100 mM Tris-Cl pH 6.8, 4% SDS, 20% glycerol, 0.02% bromophenol blue). This solution placed in boiling water for 5-10 minutes and separated using SDS-PAGE electrophoresis on a 12% Tris-Glycine. Tagged proteins are visualized by trans-illumination and the resulting images captured with CCD camera. The experimental result is provided in FIG. 11. The fluorescent bands in FIG. 11 originate from crypto-synthases.

Use of this method to identify recombinant VibB within the cell lysate of a host organism (E. coli) is shown in FIG. 11. In this example, VibB, a 32.6 kDa protein, is selectively tagged with a fluorescent reporter. Tagging was conducted by the addition of a fluorescently-tagged derivative as given in FIG. 2 and a PPTase such as the Bacillus subtilis Sfp transferase. SDS-page electrophoresis was used to separate proteins. The left frame shows fluorescence from the loading of a fluorescent tag onto VibB. The right frame shows the net protein content of the solution as stained by Coomassie blue. Lanes A-C denote synthetic appendage labels as given by A=BODIPY FL, B=N-7-dimethylamino-4-methylcoumarin and C=Oregon Green 488.

### EXAMPLE 4

Tagging of Purified Recombinant Carrier Protein Domains

Fluorescently-labeled CoA were prepared by selective modification of the free thiol of coenzyme A (FIG. 2). This CoA-DYE derivative was then incubated with heterologously expressed and purified Sfp and VibB, a small protein from the Vibrio cholera vibriobactin biosynthetic machinery containing only one carrier protein domain. Analysis was performed with SDS-PAGE, and a single fluorescent band was visualized by eye using the appropriate wavelength of light for excitation (FIG. 11). The excitation wavelength was chosen based on using the appropriate combination of excitation with UV-visible light and the appropriate cutoff filters. Coomasie staining of the gel verified the fluorescent label to be crypto-VibB (32.6 kD).

Use of this method to identify purified proteins containing at least one CP domain as shown in FIG. 11. This example demonstrates the utility of this method to fluorescently tag purified over-expressed and purified VibB, a standalone CP domain. In this example, VibB, a 32.6 kDa protein, is fluorescently-tagged. Tagging was conducted by the addition of a biotin-tagged derivative and a PPTase such as the Bacillus subtilis Sfp transferase. SDS-page electrophoresis was used to separate proteins. The left frame depicts blot arising from the binding of a Streptavidin-alkaline phosphatase conjugate to an biotin-labeled VibB. The right frame shows the net protein content of the solution, as given by staining with Coomassie blue.

Recombinant His-tagged VibB, purified by nickel chromatography (Ni-NTA His Bind® Resin, Novagen), was dialysed to a 0.6 mg/ml solution in 0.1M TRIS-HCl, pH 8.4 with 1% glycerol. A 200 uL aliquot of this solution is treated with 80 uL of the dye-CoA solution (see Preparation of modified CoA derivatives). The reaction is incubated at room temperature for 30 min in darkness. A 50 uL aliquot of a 10 mg/mL solution of bovine serum albumin (BSA) is added, and the protein is precipitated by the addition 800 uL of a 10% trichloroacetic acid solution and cooling at −20° C. for 30-60 min. The samples are centrifuged at 13,000×g for 4 minutes, and the supernatant is removed. The pellet was resuspended in 1:1 mixture of 1.0 M Tris-HCl pH 6.8 and 2× SDS-PAGE sample buffer (100 mM Tris-Cl pH 6.8, 4% SDS, 20% glycerol, 0.02% bromophenol blue). This solution placed in boiling water for 5-10 minutes and separated using SDS-PAGE electrophoresis on a 12% Tris-Glycine. Tagged proteins are visualized by trans-illumination and the resulting images captured with CCD camera. The outcome of this experiment is provided in FIG. 11.

### EXAMPLE 5

Tagging of Natively Expressed Carrier Protein Domains

Fluorescent tagging using reagents prepared in FIG. 2 was repeated on proteins from crude cell lysate from recombinant E. coli K12 cells following iron-starving conditions, which include growth in minimal nutrient media and iron chelation by growth in minimal media and addition of 2,2-dipyridyl. These conditions induce enterobactin production in the organism, which is synthesized by NRP synthase proteins EntB, EntE, and EntF. (FIG. 12). Both EntB and EntF contain carrier protein domains that can be post-translationally modified by 4′-phosphopantetheinyltransferase. Cell lysate from the iron starved cells was dialyzed to remove small molecules (<10 kDa), incubated with CoA-DYE and recombinant Sfp, and analyzed by SDS-PAGE. When viewed under irradiation, recombinant EntF and EntB are visualized as fluorescent bands that can be verified with two methods. First, standard Coomasie staining showed the fluorescent bands to have the proper molecular weight when compared to molecular weight markers. Second, bands from an unstained gel were subjected to mass spectroscopic protein sequencing (Qstar MS-MS) to reveal the sequences of EntF and EntB after searching GenBank protein databank.

E. coli K12 cells are starved of iron as follows. E. coli K12 cells in a 1 liter of Lauria-Bertani (LB) media was incubated at 37° C. to an OD of ˜0.7. The cells are treated with 2,2-dipyridyl to a final concentration of 0.2 mM and allowed to incubate an additional 4 hours at 37° C. The culture was then centrifuged, and the resuspend cell pellets was lysed by sonication at 0° C. in 30 ml of 0.1M Tris-Cl pH 8.0 with 1% glycerol in the presence of 500 uL of a 10 mM phenylmethanesulfonyl fluoride (PMSF) solution in isopropanol with 50 uL of a protease inhibitor cocktail (A mixture of protease inhibitors with broad specificity for the inhibition of serine, cysteine, aspartic and metallo-proteases, and aminopeptidases. Contains 4-(2-aminoethyl)benzenesulfonyl fluoride (AEBSF), pepstatin A, E-64, bestatin, and sodium EDTA, Sigma-Aldrich Inc.). An 80 uL aliquot of the modified-CoA solution was added to 200 uL of the cell lysate along with 30 ug of 30 mg/mL purified Sfp. The resulting mixture was incubated at room temperature for 30 min in darkness. Proteins were precipitated from this solution by the addition of 800 uL of a 10% trichloroacetic acid solution and cooling at −20° C. for 30-60 min. The samples are centrifuged at 14000×g for 4 minutes, and the supernatant is removed. The pellet was resuspended in 1:1 mixture of 1.0 M Tris-HCl pH 6.8 and 2× SDS-PAGE sample buffer (100 mM Tris-Cl pH 6.8, 4% SDS, 20% glycerol, 0.02% bromophenol blue). This solution placed in boiling water for 5-10 minutes and separated using SDS-PAGE electrophoresis on a 12% Tris-Glycine. Tagged proteins are visualized by trans-illumination and the resulting images captured with CCD camera. Blotting analysis was conducted using the biotin-CoA derivative as described in the following section (Blot Analysis). The outcome of this experiment is provided in FIG. 12.

Use of this method to identify proteins containing at least one CP domain within the cell lysate of native producer organism is shown in FIG. 12. In this example, EntB, a 32.6 kDa protein, is selectively tagged within the culture of its natural host (E. coli). SDS-page electrophoresis was used to separate proteins. Tagging was conducted by the addition of a fluorescently-tagged derivative as given in FIG. 2 and a PPTase such as the Bacillus subtilis Sfp transferase. The left frame depicts fluorescence from the loading of a fluorescent tag onto EntB. The right frame depicts the net protein content of the solution as stained by Coomassie blue. Lanes A-C denote synthetic appendage labels as given by A=BODIPY FL, B=N-7-dimethylamino-4-methylcoumarin and C=Oregon Green 488.

### EXAMPLE 6

SDS-Page Electrophoresis

SDS-page electrophoresis can be used to detect PK, NRP, and FA synthases continuing carrier proteins through protein tagging with CoA-labeled by a fluorescent dye, biotin, a carbohydrate or oligosaccharide, a peptide sequence, or another selectable moiety (FIG. 2). Here, proteins from natural or engineered organisms are tagged with the use of a 4′-phosphopantetheinyltransferase and the CoA derivative, and subsequently separated by SDS-PAGE. The separated proteins can be visible in the gel at this stage (as in the case of fluorescent tagging), or the gel can be further processed to allow visualization of the tagged proteins. Visualized pieces of the gel can be excised for protease digestion and analysis, protein sequencing via Edman degradation or mass spectrophotometric techniques, or extracted for solution-phase assays of the purified proteins. The whole gel can also be subjected to electrophoretic transfer of the proteins to a membrane or other substrate for blot analysis.

### EXAMPLE 7

Native Protein Polyacrylamide Gel Electrophoresis

This technique can be used to detect PK, NRP, and fatty acid synthases continuing carrier proteins via native protein gel electrophoresis through protein tagging with CoA-labeled by a fluorescent dye, biotin, a carbohydrate or oligosaccharide, a peptide sequence, or another selectable moiety. Here, proteins from natural or engineered organisms are tagged with the use of a 4′-phosphopantetheinyltransferase and the CoA derivative, and subsequently separated by a native protein polyacrylamide gel. The separated proteins can be visible in the gel at this stage (as in the case of fluorescent tagging), or the gel can be further processed to allow visualization of the tagged proteins. Visualized pieces of the gel can be excised for protease digestion and analysis, protein sequencing via Edman degradation or mass spectrophotometric techniques, or extracted for solution-phase assays of the purified proteins. The whole gel can also be subjected to electrophoretic transfer of the proteins to a membrane or other substrate for blot analysis.

### EXAMPLE 8

Blot Analysis

Blotting can be performed to identify proteins with carrier protein domains. It was found that PPTases such as Sfp would accept a variety of CoA derivatives for transfer onto a carrier protein, including a biotin tag, which could be visualized by electroblotting onto nitrocellulose followed by binding with streptavidin that is modified for visualization. Biotin-CoA derivative was synthesized using a variety of linked biotin tags using a method comparable to that to attach dyes (FIG. 2). The biotin-linked 4′-phosphopantetheine was successfully transferred to apo-VibB with recombinant Sfp. The biotin-tagged VibB was then identified by a blot: purified with SDS-PAGE or native protein gel, electro-transferred to nitrocellulose, and incubated sequentially with streptavidin-linked alkaline phosphatase and 5-bromo-4-chloro-3-indolyl phosphate/nitro blue tetrazolium (BCIP/NBT). The outcome of this experiment is provided in FIG. 12. The biotin-labeled VibB protein on the nitrocellulose membrane stained dark blue due to enzymatic dephosphorylation of BICP and precipitation of the dark blue product through oxidation by NBT. This assay provides convincing evidence that a biotin-streptavidin technique can also be used to purify PK and NRP synthases that contain carrier protein domains with affinity chromatography. This assay can be conducted with any affinity tag and molecular binding partner, including mannose-conconavalin A, and peptide-antibody interactions. We have reproduced these results using mannose-linked CoA tagging to VibB with Sfp, separating on SDS-PAGE, blotting to nitrocellulose, and visualizing with conconavalin-linked peroxidase and peroxidase substrate (3-Amino-9-ethylcarbazole).

One liter of E. coli BL21 (DE3) cells induced to express recombinant Vib B protein were lysed in 30 mL 1M Tris-Cl pH 8.0 with 1% glycerol in the presence of 500 uL of a 10 mM phenylmethanesulfonyl fluoride (PMSF) solution in isopropanol with 50 uL of a protease inhibitor cocktail (A mixture of protease inhibitors with broad specificity for the inhibition of serine, cysteine, aspartic and metallo-proteases, and aminopeptidases. Contains 4-(2-aminoethyl)benzenesulfonyl fluoride (AEBSF), pepstatin A, E-64, bestatin, and sodium EDTA, Sigma-Aldrich Inc.) by sonication. A 40 uL of the CoA-biotin solution was added 200 uL of cell lysate containing overexpressed Vib B and 1 uL of a 34 mg/mL solution of purified Sfp and the reaction was incubated at room temperature for 30 minutes in darkness. Proteins were precipitated from this-solution by the addition of 800 uL of a 10% trichloroacetic acid solution and cooling at −20° C. for 30-60 min. The samples are centrifuged at 14000×g for 4 minutes, and the supernatant is removed. The pellet was resuspended in 1:1 mixture of 1.0 M Tris-HCl pH 6.8 and 2× SDS-PAGE sample buffer (100 mM Tris-Cl pH 6.8, 4% SDS, 20% glycerol, 0.02% bromophenol blue). This solution placed in boiling water for 5-10 minutes and separated using SDS-PAGE electrophoresis on a 12% Tris-Glycine. Following separation, the gel was transferred to nitrocellulose and blotted.

Blots were incubated with 5% milk in TBST for 30 minutes at room temperature with shaking. The blots were then transferred directly to 10 mL of a 5% milk in TBST solution containing 10 uL of 25 mg/mL streptavidin-alkaline phosphatase conjugate (Pierce Chemical Co.) and incubated at room temperature for 1 hour. After this incubation, the blot was washed 3 times for 10 minutes with 20 mL of TBST at room temperature. Finally, the blot was incubated in 2 mL of Alkaline-phosphatase substrate solution (0.15 mg/mL BCIP, 0.30 mg/mL NBT, 100 mM Tris, 5 mM MgCl2 pH 9.5, Sigma-Aldrich Inc.) for 5 minutes or less at 37° C.

The affinity recognition technique is shown in FIG. 12. In this example, recombinant VibB has been selected using an affinity method. Tagging was conducted by the addition of a biotinylated CoA-derivative and a PPTase such as the Bacillus subtilis Sfp transferase. See FIG. 12A. FIG. 12B shows a blot verifying the ability of an biotinylated CoA-derivative to label native EntB and EntF. FIG. 12C shows a blot verifying the ability of an biotinylated CoA-derivative to label VibB. Each reaction contained 200 uL of an E. coli lysate containing approximately 0.12 ug of VibB. This blot was developed by transferring protein from a SDS-page gel onto PDVF and/or a nitrocellulose paper and developing by the sequential addition of a Streptavidin Alkaline Phosphatase conjugate followed by exposure to BCIP/NBT. (FIG. 12D) The net protein content of the solution as stained by Coomassie blue. A gradient of biotinylated-CoA derivative was been placed across the gel as given by lanes 1 with 40 μM, 2 with 20 μM, 3 with 10 μM, 4 with 5 μM, 5 with 2.5 μM, 6 with 1.25 μM, 7 with 0.624 μM, 8 with 0.312 μM, and 9 with 0.156 μM. Note that metal induction is required for the overexpression of the native EntB and EntF proteins thereby minimizing interfence when examining the overexpression of recombinant carrier proteins conventional E. coli expression vectors.

### EXAMPLE 9

Affinity Chromatography

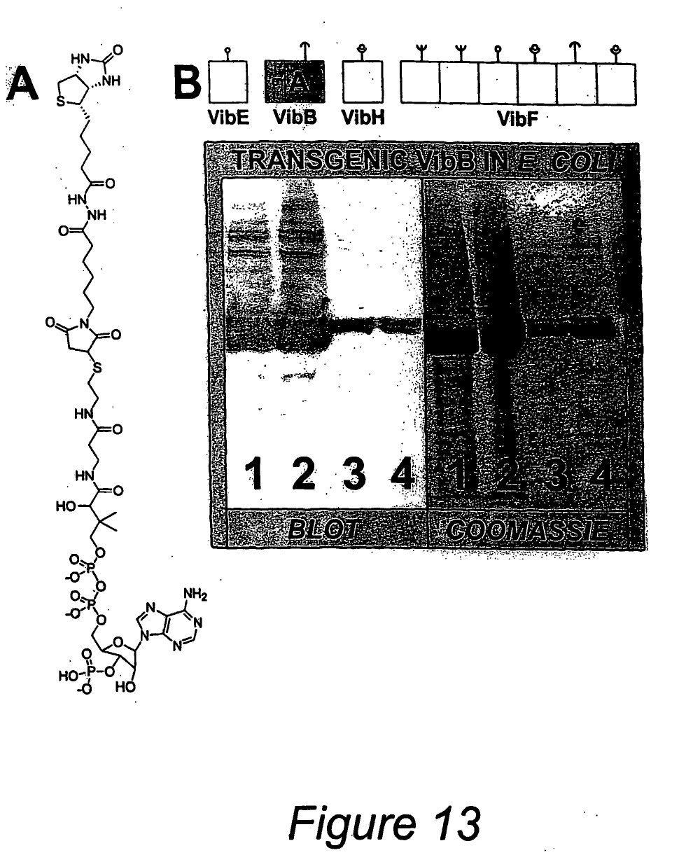

In order to isolate proteins containing at least one carrier protein domains, we reasoned that the above tagging methods can be transferred to affinity chromatography and isolation techniques. To this end, we incubated biotinylated CoA derivatives (FIG. 2) with from crude cell lysate from apo-VibB-producing E. coli (as described above) and ran the mixture over a small column loaded with streptavidin-linked-agarose resin. Following washing, some of the resin was boiled to release biotin-bound protein, and the sample was subjected to SDS-PAGE as well as a blot against streptavidin-phosphatase conjugate. Both the Coomasie-stained gel and the blot demonstrated that VibB was successfully purified with biotin affinity chromatography (FIG. 13). In addition to high affinity methods, native proteins were isolated using non-denaturing purification for instance the affinity between carbohydrate-tagged proteins (i.e. beta-mannosylated proteins) and lectin linked-agarose resins (i.e., Conconavalin A). Here, bound protein was eluted off the agarose with a gradient of carbohydrate (i.e., mannose for beta-mannosylated proteins), and the purified protein was identified with SDS-PAGE and blot against a lectin peroxidase conjugate (i.e., (i.e., Conconavalin A-peroxidase conjugate). This protocol produced pure, non-denatured VibB tagged with mannose. This protocol can be conducted with any affinity tag and molecular binding partner, including mannose-conconavalin A, peptide-antibody, and or peptide-protein interactions. We have also reproduced these results using mannose-linked CoA tagging to VibB with Sfp, isolating on conconavalin A-linked agarose column, and eluting with increasing concentrations of free mannose. This technique has the benefit of providing non-denatured protein, which can be further manipulated by enzyme activity assays to probe individual domains, modules, or full synthase activity.

A 200 uL aliquot of cell culture induced with IPTG to overexpress recombinant EntB or VibB was combined with 40 uL of biotinylated-CoA B1 and 1 uL of 11 mg/mL purified Sfp and allowed to react for 30 min at room temp in the dark. 20 uL agarose-immobilized Streptavidin (4 mg/mL Streptavidin on 4% beaded agarose) was added to each sample and incubated at 4° C. for 1 hour with constant vigorous shaking. After centrifugation at 14,000×g for 1 min, the supernatant was decanted and the samples were washed 3 times with a solution containing 100 mM Tris-Cl pH 8.4 and 1% SDS in water. After washing, the samples were boiled in 50 mL 1× SDS sample buffer for 10 min, centrifuged, and the supernatant run on a 12% Tris-Glycine gel.

Affinity purification is shown in FIG. 13. In this example, VibB has been purified from culture using either a biotinylated and/or mannosylated CoA derivatives. FIG. 13A shows a blot indicating the binding of Streptavidin. FIG. 13B shows protein content in each gel as indicated by Coomassie blue staining. Each gel depicts four lanes 1-4 developed from E. coli cell lysate contain over-expressed VibB. Lane 1, 3 and 4 were treated with 20 μM of B1 and 34 μg of Sfp per 200 μL of cell culture, while lane 2 was treated with 40 μM of B1 and 34 μg of Sfp per 200 μL of cell culture. Lanes 1-2 were developed without purification on an affinity column. Development was conducted by exposure to an excess of Streptavidin-Alkaline Phosphatase conjugate followed by exposure to BCIP/NBT. Lane 3 was purified using a column containing 10 μg of Streptavidin-agarose prior to development. Lane 4 was purified using a column containing 20 μg of Streptavidin-agarose prior to development.

### EXAMPLE 10

Removal of Tag

New tools for the tagging of proteins containing carrier protein domains for identification, isolation, and manipulation have been demonstrated. Now we further demonstrate the ability of this method by developing a tool to selectively remove these tags. This activity is useful for reconstitution of full enzyme activity after affinity purification through the above tagging technology. Once proteins containing carrier proteins have been isolated, removal of the tagged 4′-phosphopantetheine-labeled moiety can be performed in order for the carrier proteins to resume natural activity. This can be accomplished with a phosphodiesterase that cleaves the phosphate linkage between the serine of the carrier protein and the tagged pantetheine. In particular, acyl-carrier-protein phosphodiesterase (ACP-PDE), used in natural systems to remove 4′-phosphopantetheine from fatty acid acyl carrier proteins, can be used for this purpose.

### EXAMPLE 11

Kinetic Analysis

Proteins identified, cloned and/or isolated through this study can also be used to determine kinetic properties of a given synthetic system. Herein, the loading and transfer properties of identified and purified FA, PK, and NRP synthases can be determined in vitro. Such studies can be used to quantify the efficiency of a given PPTase/carrier protein pair as well as to determine the efficiency of PPTase activity with individual domains, individual modules, multiple modules, or complete biosynthetic systems. PPTase activity can be simply assayed through the fluorescent labeling technique described herein. Time course experiments can be conducted to determine kinetic measurements of K_{cat} and Kₘ values for individual carrier protein substrates or for individual fluorescent CoA derivatives. These techniques can also be used to determine kinetic constants for inhibitors of the 4′-phosphopantetheinylation process. These studies would involve time course experiments followed by protein precipitation via trichloroacetic acid or ammonium sulfate, wash, and fluorescent intensity measurement of tagged proteins. In addition, equlibrium based techniques such as equilibrium dialysis can also be used to identify the amount of reporter uptake as given by concentration of crypto-synthase. These data can yield rate information for further studies.

### EXAMPLE 12

Mechanistic Studies

Three major activities can be simply analyzed through biochemical techniques: these include (but are not limited to) posttranslational modification, amino acid or acyl monomer loading, condensation or ketosynthase, and thioesterase activity. For instance, a module isolated from a transgenic expression system and purified using mannosylated tagging, conconavalin A-agarose affinity, and untagged using a PDEase can be subsequently analyzed for in vitro 4′-phosphopantetheinylation kinetic rates with a PPTase and a fluorescent CoA derivative with a time course study. Subsequently the crypto-synthase (prepared by incubated with CoA and a PPTase) can be probed for loading in vitro: adenylation (in NRP synthase systems) or acyltransferase (in PK and FA synthase systems) activity. Here, the isolated crypto-enzymes are incubated with radiolabeled amino acids and ATP (in NRP synthase systems) or radiolabeled malonyl CoA or methylmalonyl CoA (in PK and FA synthases). These experiments can be and analyzed by SDS-PAGE and phosphorimaging to determine whether the carrier protein domain is properly loaded with the proper monomer. This experiment can also be carried out with other techniques, for instance using radiolabeled pyrophosphate with NRP synthases and isolating ATP to probe for pyrophosphate exchange. Should enzymes be properly loaded, condensation activity (for NRP systems) or ketosynthase (for PK and FA systems) can be studied next. Using radiolabeled monomers pre-loaded onto the carrier proteins, a condensation/ketosynthase reactions can be identified between modules by TCA precipitation and SDS-PAGE and phosphorimaging. Alternatively, N-acetylcystamine thioesters of monomers or oligomers can be used to probe internal condensation or ketosynthase activities in a synthase. Thioesterase activities are frequently probed with the use of N-acetylcystamine thioesters of linear precursors and analyzed for cyclization or hydrolysis activity with chromatographic and mass spectroscopy methods.

### EXAMPLE 13

Synthesis of Coenzyme A Derivatives

A library of CoA derivatives is shown in FIG. 2 and synthetic entry to this library is outlined in FIG. 1. As denoted in FIG. 1 multiple routes including a novel stepwise route as shown on the left of FIG. 1 provide facile access to derivatization of CoA. These routes permit functional modification about R1-Rn. In the synthetic scheme of FIG. 1, reactions a-e result in synthesis of phosphopantoic acid (product of e) which is achieved only through this route. Additionally, reaction m for the synthesis of a reporter-functionalized coenzyme A is achieved only through this route.

For example, the following synthetic scheme can be used.

General. All reactions were carried out under argon atmosphere in dry solvents with oven dried glassware unless otherwise noted. NMR spectra were taken on Varian 300 MHz or 400 MHz NMR machines and standardized to the NMR solvent except for ³¹P NMR, where signals were standardized to 85% H₃PO₄. Chemical shifts are reported in parts per million relative to tetramethylsilane. Silica gel chromatography was carried out with Silicycle 60 Angstrom 230-400 mesh.

[(2R, 4R)-2-(4-Methoxy-phenyl)-5,5-dimethyl-[1,3]dioxane-4-yl]methanol (6)—See literature preparation by Mukaiyama. See Shiina, I.; et al., Bull. Chem. Soc. Jpn., 74, 113-122, 2001.

[(2R, 4R)-2-(4-Methoxy-phenyl)-5,5-dimethyl-[1,3]dioxane-4-carboxylic acid (7)—Swern oxidation was carried out on 6 (5.20 g, 18.44 mmol) as per Mukaiyama's procedure for the preparation of (2R, 4R)-2-(4-Methoxy-phenyl)-5,5-dimethyl-1,3-dioxane-4-carbaldehyde and the resultant oil was purified by silica gel chromatography (6:1 to 2:1 Hexanes/EtOAc) to yield the product as a clear oil that crystallized under high vacuum (3.72 g, 75%).

The product of the preceding reaction (790 mg, 2.82 mmol) was dissolved in MeOH/water/CH₂Cl₂ (3:1:1, 50 mL). NaH₂PO₄.H₂O (778 mg, 5.64 mmol) and NaClO₂ (1.02 g, 11.28 mmol) were added, and the solution turned yellow within an hour. The reaction mixture was diluted with Ethyl Acetate (100 mL), and the organic layer was washed with water (25 mL). The aqueous washes were combined and acidified with 1M HCl, and extracted with ethyl acetate. The new organic layer was combined with the old organic layer and the mixture was washed with brine (25 mL) and dried over anhydrous sodium sulfate and evaporated in vacuo to afford 7 (350 mg, 42%) as a sticky white solid. The product was used without further purification. ¹H NMR (CDCl₃, 300 MHz) δ 7.41 (d, J=9.0 Hz, 2H), 6.90 (d, J=11.6 Hz, 2H), 5.51 (s, 1H), 4.22 (s, 1H), 3.80 (s, 3H), 3.76 (d, J=11.7 Hz, 1H), 3.66 (d, J=11.6 Hz, 1H), 1.19 (s, 3H), 1.09 (s, 3H). ¹³C NMR (CDCl₃, 100 MHz) δ 169.5, 160.2, 129.3, 127.5, 113.8, 101.6, 82.9, 78.2, 55.3, 33.1, 21.6, 19.3.

2-Tritylmercapto-ethylamine (9)—To cystamine, HCl 8 (1.50 g, 14.4 mmol) and trifluoroacetic acid (3.28 g, 2.22 mL, 28.7 mol) dissolved in CH₂Cl₂ with a drying tube, trityl chloride (4.20 g, 15.1 mmol) was added. The solution immediately turned a dark yellow color. After 30 minutes the reaction was quenched with 1 M NaOH (30 mL) turning the solution back to clear. The organic layer was diluted with CH₂Cl₂ (75 mL), and additional 1 M (20 mL) was added, and the aqueous layer was separated. The organic layer was then washed with brine (20 mL), dried over sodium sulfate, and concentrated in vacuo to give a yellow oil. The oil was purified by flash chromatography (1:4 to 1:1 MeOH/EtOAc) to give 9 (3.14 g, 63%) as a clear oil which solidified when left under vacuum overnight. ¹H NMR (400 MHz, CDCl₃) δ 7.41 (m, 6H), 7.27 (m, 6H), 7.20 (m, 3H), 2.57 (t, J=8 Hz, 2H), 2.33 (t, J=8 Hz, 2H).

3-(Fmoc-Amino)-N-(2-tritylsulfanyl-ethyl)-propionamide (10)—9 (100 mg, 0.287 mmol), Fmoc-β-Alanine (89.3 mg, 0.287 mmol), EDC (55.0 mg, 0.287 mmol), and HOBt (44 mg, 0.287 mmol) were combined and dissolved in dry THF (10 mL). DIPEA (70 μL) was added, and the reaction was allowed to stir for 4.5 hours. The reaction was quenched with water and diluted with diethyl ether (20 mL). The organic layer was washed with water (5 mL), brine (5 mL), dried over anhydrous sodium sulfate, and evaporated in vacuo. The resultant oil was purified by column chromatography (1:1, Hexanes:EtOAc) to yield 10 (126 mg, 68%) as a sticky white solid. ¹H NMR (300 MHz, CDCl₃) δ 7.73 (d, J=7.5Hz, 2H), 7.55 (d, J=7.5 Hz, 2H), 7.45-7.15 (m, 19H), 5.46 (b, 2H), 4.33 (d, J=7.2 Hz, 2H), 4.17 (t, J=6.6 Hz, 1H), 3.42 (m, 2H), 3.06 (q, J=6.3 Hz, 2H), 2.41 (t, J=6.0 Hz, 2H), 2.30 (t, 2H). ¹³C NMR (100 MHz, CDCl₃) δ 170.0, 156.3, 144.3, 143.7, 141.1, 129.3-126.7 (multiple signals), 125.0, 119.8, 66.7, 47.3, 38.2, 35.9, 31.9. m/z found: 635.12 amu. [M+Na]⁺ calcd. C₃₉H₃₆O₃N₂SNa⁺: 635.23 amu.

2-(4-Methoxy-phenyl)-5,5-dimethyl-[1,3]dioxane-4-carboxylic acid [2-(2-tritylsulfanyl-ethylcarbamoyl)-ethyl]-amide (12)—10 (44 mg, 0.068 mmol) was dissolved in DMF (5 mL), and piperidine was added (1 mL). The DMF and piperidine were evaporated under reduced pressure, and to the dry residue (crude 11) EDC (13 mg, 0.068 mmol), HOBt (9 mg, 0.068 mmol), and 7 (20 mg, 0.068 mmol) were added. The flask was evacuated and filled with argon. The contents were dissolved in THF and DIPEA (18 mg, 0.024 mL, 0.136 mmol) was added. The reaction was allowed to stir overnight and it was quenched with saturated ammonium chloride and diluted with diethyl ether (25 mL). The organic layer was separated and washed with water (5 mL), brine (10 mL), dried over anhydrous sodium sulfate, and evaporated in vacuo. The product was purified by silica get chromatography (1:1 to 1:5 Hexanes/EtOAc) to give 12 (20 mg, 47%) as a clear film. It should be noted that in this form the product will slowly deprotect in chloroform to give S-trityl pantetheine. ¹H NMR (CDCl₃, 400 MHz) δ 7.40-7.37 (m, 6H), 7.28-7.26 (m, 6H), 7.21-7.18 (m, 5H), 6.97 (t, J˜8 Hz, 1H), 6.89 (d, J=8.8 Hz, 2H), 5.76 (t, J˜8 Hz 1H), 5.39 (s, 1H), 4.02 (s, 1H), 3.79 (s, 3H), 3.67 (d, J=12 Hz, 1H), 3.60 (d, J=12 Hz, 1H), 3.48 (d, J=6 Hz, 1H), 3.45 (d, J=6 Hz, 1H), 3.02 (8-plet, J=6.4 Hz, 1H), 2.98 (8-plet, J=6.0 Hz, 1H), 2.385 (t, J=6.8 Hz, 1H), 2.378 (t, J=6.4 Hz, 1H), 2.31 (t, J=6.0 Hz, 2H), 1.05 (s, 3H), 1.04 (s, 3H). ¹³C NMR (CDCl₃, 100 MHz) δ 170.6, 169.5, 144.6, 130.1, 129.5-126.6 (multiple signals), 113.7, 101.2, 83.8, 78.4, 66.8, 55.3, 38.2, 35.9, 34.8, 33.0, 31.7, 21.8, 19.1. ¹H-COSY couplings δ 6.97-3.46, 5.76-3.00, 3.46-2.31, 3.00-2.38.

Pantetheine (3)—12 (13 mg, 0.020 mmol) was dissolved in methanol (2 mL), and a 0.1 M solution of iodine in methanol (2 mL) was added. After 20 minutes, zinc metal was added to remove the iodine. The solution was filtered through celite and evaporated in vacuo. The remaining residue was purified twice by silica gel chromatography (1:1 MeOH/EtOAc) to remove iodine salts from the product 3 (5 mg, ˜85%). ¹H NM (D₂O, 400 MHz) δ 4.00 (s, 1H), 3.51-3.54 (m, 5H), 3.40 (d, J=11.2 Hz, 1H), 2.87 (t, J=6.0 Hz, 2H), 2.53 (t, J=6.4 Hz, 2H), 0.94 (s, 3H), 0.90 (s, 3H). m/z found 577.22, calcd. C₂₂H₄₂O₈N₄S₂Na⁺: 577.23

(R)-3-Benzyloxy-4,4-dimethyl-dihydro-furan-2-one (13)—Silver oxide (3.54 g, 15.3 mmol) and benzyl bromide (1.4 g, 8.4 mmol) were added to a solution of D-Pantolactone (1.0 g, 7.7 mmol) in dry DMF (25 mL) at 0° C. under nitrogen. The mixture was stirred at 0° C. for 2 h, then warmed to r.t. and stirred for an additional 20 h. The solution as diluted with dichloromethane (100 mL) and filtered. The filtrate was concentrated in vacuo, diluted with ethyl acetate, and washed with 0.5 N HCl, water, and brine. The solvent was removed in vacuo and then excess benzyl alcohol was removed by co-evaporation with water under reduced pressure to give a crystalline solid. The product was recrystallized from hexanes to give 13 (1.46 g, 86%) ¹H NMR (300 MHz, CDCl₃) δ 7.30-7.36 (m, 5H), 5.02 (d, J=12.0 Hz, 1H), 4.73 (d, J=12.3 Hz, 1H), 3.97 (d, J=9.0 Hz, 1H), 3.85 (d, J=8.7 Hz, 1H), 3.71 (s, 1H), 1.12 (s, 3H), 1.08 (s, 3H). ¹³C NMR (100 MHz, CDCl₃) δ 175.4, 137.2, 128.4, 127.98, 127.97, 80.4, 76.4, 40.3, 23.2, 19.3. Note: this product has been synthesized previously using benzyl chloride by a different method which required base; however, the optical purity was reduced even with the mild base Cs₂CO₃. Optical purity is preserved in this procedure and can be confirmed by the generation of only two diastereomers in the proceeding step which vary only at the anomeric carbon. See Dueno, E. E.; et al., Tetrahedron Lett., 40, 1843-1846, 1999.

(R)-3-Benzyloxy-4,4-dimethyl-tetrahydro-furan-2-ol (14)—To a stirred solution of 13 (3.00 g, 13.6 mmol) in dichloromethane (50 mL) at −78° C., DIBAL-H (1 M in hexanes, 16.3 mL, 16.3 mmol) was added over 30 minutes. After 2 hours, the reaction was quenched slowly at first with 60 mL of a 1:1 diethyl ether/1 M H₂SO₄ mixture. The reaction was then diluted with ethyl acetate (100 mL) and the organic layer was washed with 100 mL 1 M H₂SO₄, 10 mL of NaHCO₃(sat), 10 mL of water, and twice with 20 mL of brine. The organic phase was then dried with Na₂SO₄, and concentrated in vacuo. The crude oil was purified by flash chromatography (2:1 Hexanes/EtOAc to pure EtOAc) to yield 14 (2.85 g, 94%) as a clear oil that solidified to white clumps after it was removed from the freezer and disturbed. The product turned out to be an inseparable mixture of anomers in an approximate 2:3 ratio. ¹H NMR (CDCl₃, 400 MHz) δ 7.34-7.32 (m, 5H), 5.46 (m, 3/5H), 5.36 (d, J=2.8 Hz, 2/5H), 4.70 (d, J=12.0 Hz, 2/5H), 4.66 (d, J=11.6 Hz, 3/5H), 4.61 (d, J=11.2 Hz, 3/5H), 4.57 (d, J=12.0 Hz, 2/5H), 3.98 (b, 3/5H), 3.81 (d, J=8.4 Hz, 2/5H), 3.71 (d, J=8.0 Hz, 3/5H), 3.63 (d, J=8.4 Hz, 2/5H), 3.52 (d, J=2.8 Hz, 2/5H), 3.46 (d, J=4.0 Hz, 3/5H), 3.41 (d, J=8.4 Hz, 3/5H), 1.12 (s, 9/5H), 1.12 (s, 6/5H), 1.11 (s, 6/5H), 1.07 (s, 9/5H). ¹³C NMR (CDCl₃, 100 MHz) δ 127.5-128.7, 103.1, 97.8, 91.8, 85.6, 76.7, 79.1, 74.7, 72.7, 42.5, 26.1, 24.4, 20.8, 20.1. m/z found 245.07, [M+Na⁺] calcd. C₁₃H₁₈O₃Na⁺=245.12 amu

(E,Z)-(S)-3-Benzyloxy-2,2-dimethyl-5-phenyl-pent-4-en-1-ol (15)—To a stirred solution of benzyl triphenylphosphonium bromide (1.21 g, 2.78 mmol) in THF (15 mL) at −78° C., potassium t-butoxide (1 M in THF, 2.69 mL, 2.69 mmol) was added. The solution immediately turned orange, and was allowed to stir as it turned to crimson-orange. After 30 minutes, a solution of 9 (206 mg, 0.928 mmol) in THF was cannulated into the stirring ylide, and the reaction was allowed to warm to room temperature. After two hours, the reaction was driven to completion by heating to reflux for 40 minutes. The reaction was quenched with NH₄Cl(sat) (3 mL), diluted with diethyl ether (50 mL) and the organic layer was washed with water (10 mL) and brine (10 mL) whereupon it was dried with Na₂SO₄ and concentrated in vacuo until a yellow oil remained. The compound was purified by flash chromatography (8:1 to 4:1 Hex:EtOAc) and concentrated to a clear yellowish oil (260 mg, 95%). The product was a mixture of regioisomers that was about 3:2 E/Z. ¹H NMR (300 MHz, CDCl₃) δ 7.45-7.20 (m, 10H^{E&Z}), 7.10-7.08 (m, 2H^{E&Z}), 6.83 (d, J=12.0 Hz, 1H^{Z}), 6.54 (d, J=15.9 Hz, 1H^{E}), 6.19 (dd, J=16.2, 8.4 Hz, 1H^{E}), 5.71 (dd, J=12.0, 10.8 Hz, 1H^{Z}), 4.64 (d, J=11.7 Hz, 1H^{E}), 4.55 (d, J=11.7 Hz, 1H^{Z}), 4.34 (d, J=11.7 Hz, 1H^{E}), 4.28 (d, J=11.1 Hz, 1H^{Z}),4.11 (d, J=11.7 Hz, 1H^{Z}), 3.80 (d, J=8.4 Hz, 1H^{E}), 3.58 (d, J=10.9 Hz, 1H^{E}), 3.54 (d, J=10.9 Hz, 1H^{Z}), 3.40 (d, J=11.1 Hz, 1H^{E}), 3.33 (d, J=11.1 Hz, 1H^{E}), 0.91-0.94 (m, 6H). ¹³C NMR (100 MHz, CDCl₃) δ 137.91, 137.85, 136.6, 134.5, 126.3-128.8 (many signals), 87.67, 87.63, 71.4, 70.5, 70.0, 39.35, 39.31, 22.84, 22.79, 20.1, 19.9. m/z found 319.08, [M+Na]⁺ calcd. C₂₀H₂₄O₂Na⁺: 319.18 amu

(R)-2-Benzyloxy-4-(bis-benzyloxy-phosphoryloxy)-3,3-dimethyl-butyraldehyde (16)—To a stirred suspension of tetrazole (27 mg, 0.38 mmol) in CH₂Cl₂ (5 mL) at room temperature, N,N-diisopropyl-O,O′-dibenzyl phosphoramidite (131 mg, 127 μL, 0.38 mmol) was added. After 15 minutes, 15 dissolved in CH₂Cl₂ (2 mL) was cannulated into the stirring solution. After 2.5 hours, the solution was diluted with CH₂Cl₂, washed with water (5 mL), brine (5 mL), and dried over anhydrous Sodium Sulfate, and evaporated in vacuo. The residual oil was redissolved in a solution of CH₂Cl₂/MeOH (9:1, 5 mL), cooled to −78° C., and ozone was bubbled through the solution for 3 minutes. Dimethyl sulfide (1 mL) was added, and white vapor evolved in the flask. The flask was then removed from the −78° C. bath, and the solvent was evaporated in vacuo. Purification followed by flash chromatography (2:1 to 1:2 Hexanes/EtOAc) to yield aldehyde 16 (113 mg, 62%) as a clear viscous oil. ¹H NMR (400 MHz, CDCl₃) δ 9.66 (d, J=2.8 Hz, 1H), 7.34-7.23 (m, 15H), 5.04-4.99 (m, 4H), 4.55 (d, J=11.2 Hz, 1H), 4.40 (d, J=11.6 Hz, 1H), 3.87 (dd, J=9.6, 4.4 Hz, 1H), 3.80 (dd, J=9.6, 4.4 Hz, 1H), 3.46 (d, J=2.8 Hz, 1H), 0.95 (s, 3H), 0.94 (s, 3H). ¹³C NMR (100 z, CDCl₃) δ 203.6, 137.0, 135.6 (d, J=6.8 Hz), 127.7-128.5 (multiple signals), 86.4, 73.1, 72.1 (d, J=6.1 Hz), 69.3 (d, J=5.3 Hz), 39.8 (d, J=8.3 Hz), 21.5, 19.8. ³¹P NMR (121.4 MHz, CDCl₃) δ-1.15 ppm.

(R)-2-Benzyloxy-4-(bis-benzyloxy-phosphoryloxy)-3,3-dimethyl-butyric acid (17)—Aldehyde 16 (74 mg, 0.15 mmol) was dissolved in MeOH/CH₂Cl₂/H₂O (6:3:2, 5 mL). NaH₂PO₄ (83 mg, 0.60 mmol) was added followed by 80% NaClO₂ (34 mg, 0.30 mmol). The solution turned green after 10 minutes. After 3.5 hours, the reaction was complete by TLC (1:2 Hexanes/EtOAc). The reaction was quenched with 1 M HCl (1 mL) and the volatile solvents were evaporated in vacuo. The remaining material was extracted with CH₂Cl₂ (3×, 30 mL), and the organic extractions were combined washed with brine (10 mL), dried over anhydrous sodium sulfate, and evaporated under reduced pressure to yield 17 as a clear oil (75 mg, 99%). The product was used and characterized without further purification. It should be noted that the NMR is pH sensitive, and reported spectra were taken immediately after extraction. Further manipulation can cause some peaks to shift relative positions. ¹H NMR (400 MHz, CDCl₃) δ 7.33-7.24 (m, 15H), 5.00-4.97 (m, 4H), 4.58 (d, J=11.2 Hz, 1H), 4.35 (d, J=10.8 Hz, 1H), 3.93 (dd, J=9.6, 4.8 Hz, 1H), 3.80 (dd, J=10.0, 4.8 Hz, 1H), 3.80 (s, 1H), 0.99 (s, 3H), 0.95 (s, 3H). ¹³C NMR (100 MHz, CDCl₃) δ 173.0, 136.9, 135.7, 128.9-128.0 (multiple signals), 81.6, 72.6 (d, J=6.1 Hz), 69.6-69.3 (m), 73.2, 38.9 (d, J=8.4 Hz), 21.3, 20.0.

Phosphoric acid dibenzyl ester (R)-3-benzyloxy-2,2-dimethyl-3-[2-(2-tritylsulfanyl-ethylcarbamoyl)-ethylcarbamoyl]-propyl ester (18)—10 was deprotected by treatment with 20% piperidine in DMF (5 mL). Once the deprotection was apparent by TLC (1:2 Hexanes/EtOAc), the mixture was concentrated and evaporated under vacuum until there was no remaining piperidine or DMF. The crude film of 11 was taken to the next step without further treatment.

EDC (19 mg, 0.098 mmol), and HOBt (15 mg, 0.098 mmol) were dissolved in THF (3 mL), and in separate flasks 17 (49 mg, 0.098 mmol) and 11 (78 mg, 0.187 mmol), were dissolved in THF (2 mL each). The solution of 17 was cannulated into the flask with EDC and HOBt, followed by cannulation of the solution containing 11. DIPEA (100 μL) was then added and all of the solids within the flask dissolved. The reaction was allowed to stir for 23 hours before quenching with water. The solution was diluted with diethyl ether to 50 mL, the aqueous layer was removed, and the organic was washed with 1 M HCl (5 mL), NaHCO₃₍ₛₐₜ₎ (10 mL), and brine (10 mL). The organic layer was dried over anhydrous sodium sulfate and concentrated in vacuo. The resultant film was azeotroped twice with 25 mL MeOH and purified by silica gel chromatography (1:1 Hexanes/EtOAc to pure EtOAc). 18 (41 mg, 47%) was obtained as a clear film. ¹H NMR (300 MHz, CDCl₃) δ 7.40-7.15 (m, 30H), 7.00 (t, J=6.6 Hz, 1H), 5.71 (t, J˜6 Hz, 1H), 4.99 (t, J=7.8 Hz, 4H), 4.39 (d, J=10.8 Hz, 1H), 4.27 (d, J=10.8 Hz, 1H), 3.93 (dd, J=9.6, 4.5 Hz, 1H), 3.72 (dd, J=9.6, 4.5 Hz, 1H), 3.62 (s, 1H), 3.46 (q, J˜6 Hz, 2H), 3.00 (q, J˜6 Hz, 2H), 2.35 (t, J=6.6 Hz, 2H), 2.27 (t, J=6.9 Hz, 2H), 0.93 (s, 3H), 0.83 (s, 3H). ¹H-COSY couplings δ 7.00-3.46, 5.71-3.00, 4.394.27, 3.93-3.72, 3.46-2.27, 3.00-2.35. ¹³C NMR (100 MHz, CDCl₃) δ 170.6, 170.4, 144.4, 136.7, 129.4-126.6 (multiple signals), 83.3, 73.6, 73.0 (m), 69.2(m), 38.9 (d, J=9.1 Hz), 38.3, 35.6, 35.0, 31.8, 21.2, 20.1. ³¹P NMR (121 MHz, CDCl₃) δ-1.45 ppm.

Phosphopantetheine (2)—Napthalene (271 mg, 2.10 mmol) in THF (2 mL), was added to lithium metal (15 mg, 2.2 mmol) that had been rinsed with dry hexanes. After 30 minutes, a dark green color evolved which turned so dark it appeared black 1 hour after addition of naphthalene. After 1.25 hours, the solution was cooled to −20° C. in an isopropanol/dry ice bath, and 18 (25 mg, 0.028 mmol) in THF (3 mL) was added by cannula. The solution turned from black to light red immediately. After 2 more hours water (2.5 mL) was added to the solution which removed all color. More water was added (5 mL), and the solution was washed with CH₂Cl₂ (4×, 20 mL) and 1× with diethyl ether (15 mL). Extra solvent was evaporated, and then the aqueous layer was lyopolized. After lyopolization, a yellow solid remained, and this solid was passed through a small column of acid form AG-50W-X8 ion exchange resin, and the eluant was immediately passed through a column of Na⁺ loaded AG-50W-X8 ion exchange resin. The eluant was lyopolized to give 2 (10 mg, 90±5%) as a white sticky solid. ¹H NMR (400 MHz, D₂O) δ 4.12 (s, 1H), 3.75 (dd, J=10.8, 6.8 Hz, 1H), 3.52 (m, 4H), 3.40 (dd, J=10.0 5.2 Hz, 1H), 2.86 (t, J=10.8 Hz, 2H), 2.53 (t, J=10.4 Hz, 2H), 1.00 (s, 3H), 0.84 (s, 3H). ³¹P NMR (D₂O, 121 MHz) δ 4.50 ppm. Note that the spectra of Phosphopantetheine are pH sensitive. See Lee, C.; Sarma, R. H. J. Am. Chem. Soc., 97: 1225-1235, 1975.

### EXAMPLE 14

Combinatorial Library Analysis

New tools for the identification, sequencing, characterization, and isolation of FA, PK and NRP synthases bearing one or more than one carrier protein domain have been demonstrated. These methods can also be extended into a combinatorial screening program, therein providing access to high throughput. The construct of this combinatorial system is outlined in FIGS. 2 and 17.

Non-natural CoA derivatives can be synthesized to contain derivatives of the natural CoA molecule with variant moieties at key locations on the molecule. For instance, a library of derivatized functionality at backbone carbons within the panothenate, beta-alanine, and cystamine sub-groups of pantetheine can be created. FIG. 2 depicts the structure of Coenzyme A analogs that can be prepared. These derivatives can contain variation within the functionality within the pantetheine backbone as given by R₁-R₁₁. Modifications about R₁-R₁₁ can include the appendage of alkyl, alkoxy, aryl, aryloxy, hydroxy, halo, and/or thiol groups. In addition to backbone modifications, derivation can appear within the choice of reporter or tag. As illustrated in FIG. 2, this modification occurs about a linker and reporter. These modifications can include multimeric derivatives, including but not limited to functional groups that contain more than one fluorescent or affinity reporter and/or a combination of fluorescent and affinity reporters. Ideally each member of this library should either contain a fluorescent reporter or express an affinity that can bind to a material containing a fluorescent reporter.

Collections of the derivatives in FIG. 2 are then assembled into a library. This library is referred to herein as a library of multicolored coenzyme derivatives, as indicated in Step 1 of FIG. 17. Once prepared this library is nested in a library of different PPTases as shown by Steps 2-3 in FIG. 17. This nested library now displays combinations of the multicolored coenzyme library with different PPTases. A sample of cell culture obtained from an organism or collection of organisms of study is then added to each vessel within this library and incubated as shown in Step 4 of FIG. 17. Upon completion of incubation and isolation of protein, the activity within each reaction or vessel within this nested library is then prescreened for protein containing a fluorescent tag or reported (STEP 5). Vessels positive for the presence of a fluorescently tagged protein identified in STEP 5 of FIG. 17 are then purified through STEP 6 of FIG. 17 using SDS-page or comparable electrophoresis, and sequenced. Sequence analysis is performed in STEP 7 of FIG. 17. The sequence of proteins identified with a fluorescent tag can then be translated into an complementary oligonucleotide sequence. This sequence and portions therein can be used to clone the corresponding genes from their natural host.

A library of CoA derivatives is shown in FIG. 2 and synthetic entry to this library is outlined in FIG. 1. As denoted in FIG. 1 multiple routes including a novel stepwise route as shown on the left of FIG. 1 provide facile access to derivatization of CoA. These routes permit functional modification about R1-Rn.

A system for combinatorial screening of carrier protein (CP) domains is shown in FIG. 17. STEP 1: a library of CoA derivatives is synthesized based on the structures shown in FIG. 2. This library is then displayed within a two dimensional matrix. One matrix is made for each member of the PPTase library. STEP 2: 4′-phosphopantetheinyl transferase are relatively small enzymes (about 600 bp), and as such they can be synthesized de novo. Utilizing current in vitro evolution and gene shuffling techniques, natural and non-natural homologs of known PPTases can be synthesized and cloned into a library of plasmids for expression in E. coli. STEP 3; a nested library is constructed inserting libraries of the multicolored coenzymes into the PPTase library. This generates a 6×6 matrix wherein each unit in the matrix contains a single PPTase and a library of multicolored coenzymes. STEP 4: Cell lysates are prepared. The addition of phosphatase and protease inhibitor cocktails can be used to increase the stability protein product. DNAase can be added to decompose DNA, and proteins can be partially purified through dialysis. Dialysis can also be used to collect specific sizes of protein. In particular, 30,000, 50,000 and 100,000 MWCO dialysis provides an effective step in improving the yield of large molecular weight synthases. STEP 5: Samples of the cell lysate produced in step 4 are added to each vessel in the nested library prepared in step 3, and incubated. STEP 6: After incubation and processing of the proteins, each reaction vessel is prescreened for fluorescent protein. The presence of fluorescent protein indicates positive transfer of color from the coenzyme to a carrier protein. STEP 7: vessels that contain fluorescent protein are purified using SDS-page. STEP 8: The purified proteins from step 7 are sequenced using a combination of mass spectral, digestion, and sequence analysis.

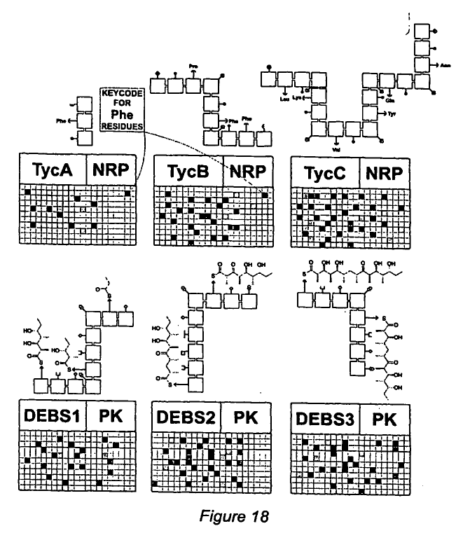

Application of these methods can be used to profile protein structure and function. The outcome of experiments conducted using single assays, libraries or microarrays can be pooled to characterize given proteins using conventional profiling algorithms (references). FIG. 18 illustrates an exemplary output from a profiler. Here individual responses to given conditions are used to identify a given biosynthetic protein. As shown, the level and position of these conditions are illustrated by two dimensional array of colored pixels. Each pixel serves to depict the activity of a given combination of carrier protein, modified coenzyme, synthetic appendage label and processing enzyme (i.e., PPTase, nucleotidase and/or ACP-PDEase).

New tools for tagging, analysis, and manipulation of FA, PK and NRP biosynthetic enzymes with a selective and powerful catalytic system have been demonstrated. The analytical methods herein can be used to analyze protein solubility, proper folding, and post-translational modification ability of engineered biosynthetic systems. The isolation techniques can be utilized as a means to purify unknown proteins with carrier protein activity in known and unknown biosynthetic systems.

### EXAMPLE 15

Synthesis of CoA-Reporter Analogs

Given the assumption that PPTases would accept substrates other than thioesters, analogs of CoA were created that would require simple preparation and purification. To this end maleimides were chosen for their specific reactivity with sulfhydryl groups. Michael attack of the thiol in CoA onto a maleimide-linked reporter molecule would result in selective and irreversible covalent attachment. Trievel R. C., et al., Anal. Biochem., 287: 319-328, 2000. Unreacted malemide-reporter could then be removed by organic wash or with the use of a thiol-terminating scavenger resin. To investigate the feasibility of this approach (FIG. 2), several CoA derivatives were synthesized (2) with the use of fluorescent-labeled and affinity reporter-labeled maleimides (Table 1). Commercially available fluorescent maleimide 1a (BODIPY® FL N-(2-aminoethyl)maleimide) was first used to yield analog 2a. Unreacted la was extracted from the media using ethyl acetate. Thin layer chromatography was used to demonstrate completion of the reaction and successful extraction of unreacted maleimide. The same procedure was followed with Oregon Green® 488 maleimide 1b and N-(7-dimethylamino-4-methylcoumarin-3-yl)maleimide 1c. Affinity reporters were also synthesized. Biotin maleimides 1d and 1e were coupled to CoA in the same manner as the fluorescent dyes above, except thiol-terminating scavenger resin was used for extraction of the unreacted maleimides. α-Mannosyl maleimide is not soluble in organic solvents; therefore scavenger resin extraction is also used with this reporter.

TABLE 1
Fluorescent-Labeled and Affinity Reporter-Labeled Maleimides
FLUORESCENT REPORTERS

| | |
|---|---|
| 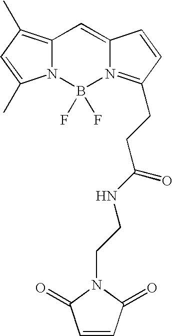 | 1a |
| 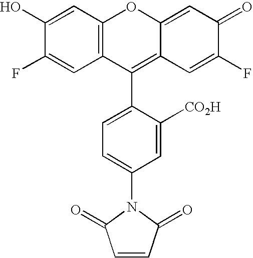 | 1b |
| 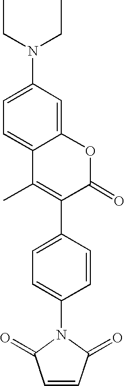 | 1c |

AFFINITY REPORTERS

| | |
|---|---|
| 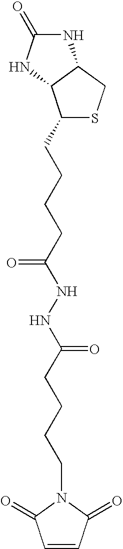 | 1d |
| Receptor Protein: Avidin/Streptavidin | |
| 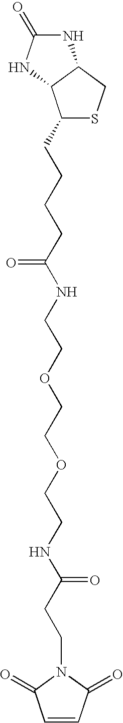 | 1e |
| Receptor Protein: Avidin/Streptavidin | |
| 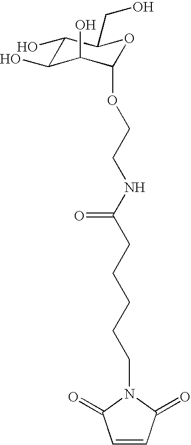 | 1f |
| Receptor Protein: | |
| Concanavalin | |
| Mannose-Binding | |
| Protein | |

### EXAMPLE 16

PPTases Can Selectively Transfer Fluorescent CoA Derivatives to Carrier Proteins

To investigate PPTase transfer of non-thioester CoA derivatives, Sfp was used for post-translational modification of known, heterologously expressed CP domains (FIG. 11). As a first experiment, VibB was used. VibB is a small protein from the Vibrio cholera vibriobactin biosynthetic machinery that consists of a modular NRP synthase system. VibB contains only one carrier protein domain and as such is a perfect model system due to its small size and facile expression in E. coli. Cell lysate was collected from induced E. coli BL21 cells producing VibB from a pET24 expression vector. An aliquot of this lysate was incubated with CoA-BODIPY derivative and recombinant Sfp and analyzed by SDS-PAGE. When viewed under UV irradiation, recombinant VibB was visualized as a fluorescent band (FIG. 11C). Coomassie staining of the gel confirmed the band to be fluorescently-tagged VibB (32.6 kD) (FIG. 11C). Similarly, the formation of other fluorescent reporters were tested. Comparable labeling apo was obtained after repetition of this experiment with Oregon Green® 488 maleimide (Table 1, 1b) and N-(7-dimethylamino-4-methylcoumarin-3-yl)maleimide (1c). Further proof was obtained by sequence analysis. A gel identical to FIG. 11C was electrophoretically transferred to a polyvinylidene fluoride membrane, and the fluorescent band corresponding to VibB was excised from the membrane. The resulting piece was subjected to N-terminal amino acid sequencing by Edman degradation. Edman P., Acta Chem. Scand., 4: 283-293, 1950. The first 10 amino acids of the returned sequence, “MAIPKIASYP”, mapped to the correct protein, VibB, when searched with BLAST against 1.4 million sequences in GenBank. All three fluorescent analogs could be used to label, visualize, isolate, and sequence VibB.

Since Sfp has been shown to 4′-phosphopantetheinylate both modular and iterative NRP and PK synthases, carrier protein labeling on other systems was demonstrated. Since iterative systems like type II PK carrier proteins comprise a major group of PK synthases (1), ACPs from three different type II PK producer strains were chosen: frenolicin (fren) from Streptomyces roseofulvus, oxytetracycline (otc) from S. rimosus, and tetracenomycin (tcm) from S. glaucescens. These proteins were heterologously expressed in E. coli BL21 cells from pET22 vectors. Cell lysate from IPTG-induced cultures was treated with 2a and recombinant Sfp and separated on SDS-PAGE. Each of these carrier proteins was labeled as 3a and identified by comparing the uptake of fluorescence versus Coomassie staining in FIG. 11C.

### EXAMPLE 17

Fluorescent Labeling of Carrier Protein Domains Can be Used to Quantify Post-Translational Modification in Engineered Systems

For metabolically-engineered systems, carrier proteins become active only after post-translational modification. This modification can be conducted either by PPTases endogenous to the heterologous host or by the co-expression of a PPTase, often under low-level gene expression. Kao C. M., et al., Science, 265: 509-512, 1994; Bedford D. J., et al., J. Bacteriol., 177: 4544-4548, 1995. The fluorescent CP domain labeling technique provides a robust and useful means to compare the in vivo activity of native and differentially expressed heterologous PPTases. By fluorescently tagging unmodified CP domains in cell lysate, purifying the protein, and spectrophotometrically comparing fluorescently tagged protein versus total protein, one can quantify the amount of in vivo post-translationally modified protein. In this manner different promoters may be compared and optimized. This technique was demonstrated with a common co-expression system, whereby the CP domain was expressed in a pET vector (with a T7 promoter) and the PPTase was expressed in a pREP4 vector (with a lacI promoter). A small CP domain, TcmACP, was inserted in a pET22 vector, both with and without co-expressed Sfp in a pREP4 vector.

To determine the relative activity of co-expressed Sfp, a set of cultures of BL21(DE3) E. coli were transformed with tcm ACP, and a subset were co-transformed with sfp. The cells were harvested at several post-induction time points. The cell lysates were treated with an excess of CoA-BODIPY derivative, and a subset was treated with additional recombinant Sfp to compare in vitro activity of co-expressed PPTase. The Tcm ACP in each sample was purified by nickel chromatography with EDTA elution. Purified protein was then analyzed for relative fluorescent intensity as a function of total protein concentration, and these results were tabulated to reveal amount of in vitro labeling in the engineered system. Here carrier proteins unmodified in vivo were fluorescently tagged in the cell lysate. This experiment indicates that Sfp insufficiently tags Tcm ACP when expressed at a low level prior to induction by IPTG. Here, the lac promoter allows basal levels of expression (“leaky” expression) that results in nearly 50% unmodified Tcm ACP. However protein concentration at this time point (time=0) is 5- to 10-fold lower than at maximal production levels. After induction, 4′-phosphopantetheinylation of the CP follows a time-dependent lag, reaching a maximum at 3 hours post-induction with just 4% unmodified protein. This system is sufficient for production of modified CP domains under high expression.

This study offers a means to evaluate transcriptional regulation as it applies to post-translational modification of biosynthetic enzymes. Both promoter level and gene copy number are important for metabolic engineering efforts, and post-translational modification must be optimized. Jones K. L., et al., Metab Eng., 2: 328-338, 2000. Selective use of promoters to control these events are important to the production of active enzyme and downstream products.

### EXAMPLE 18

Carrier Protein Western Blot

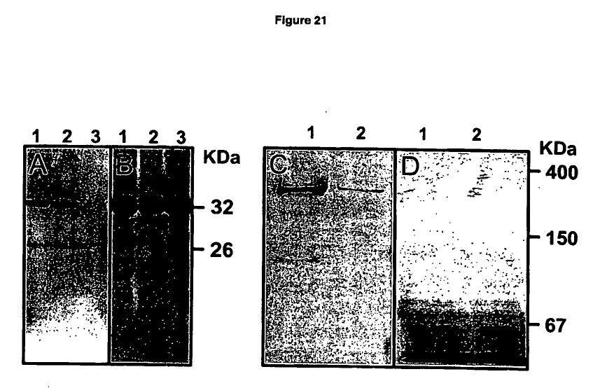

While fluorescent techniques can be used to identify proteins by direct visualization with very low expression (25 μg/L), where the Coomassie stained gel indicated little to no protein present, more sensitive reporter systems were examined. It was found that Sfp would also accept biotinylated derivatives, therein allowing protein identification by Western blotting. Towbin H., et al., Biotechnology, 24: 145-149, 1979. To this end, biotinylated CoA analogs from N-biotinoyl-N′-(6-maleimidohexanoyl)hydrazide (1d) and biotinyl-3-maleimidopropionamidyl-3,6-dioxaoctanediamine (1e), respectively, were prepared. Aliquots containing these biotinylated CoA analogs were incubated with Sfp and cell lysate from apo-VibB-producing E. coli. Following SDS-PAGE, the gel was electrotransferred to nitrocellulose and incubated sequentially with streptavidin-linked alkaline phosphatase and 5-bromo-4-chloro-3-indolyl phosphate/nitro blue tetrazolium (BCIP/NBT) (FIG. 21A). Here carrier protein could be detected at a limit of 100 pg/lane (or 5 ng/ml). Some conditions were encountered that yielded a high background level due to the labeling of native E. coli proteins. To counter this effect, diluting biotinylated CoA analogs with unmodified CoA lowered the background and increased the selection of carrier proteins within E. coli cell lysate (FIG. 21A, lane 3). This experiment qualitatively illustrates that Sfp accepts both CoA and its biotinylated CoA analogs with comparable efficiency. Additionally, the effects of tether length were examined.

This technique also imparts modest utility in identifying CP domains from the lysate of native cultures. While natural product producer strains express PPTases sufficient for post-translational modification of their native carrier proteins, a small percentage of unmodified sites remain after cell lysis. These unmodified CP domains can still be used for in vitro reporter tagging with Sfp for protein visualization. Western blotting of PK synthase enzymes has been demonstrated using the 6-deoxyerythronolide B synthase (DEBS) system from Saccharopolyspora erythraea and polyclonal antibodies raised against the recombinant proteins. Caffrey P., et al., FEBS Lett., 304: 225-8, 1992. The DEBS system was used for the first native CP tagging experiments. A type I modular PK synthase, DEBS represents a class of synthases in which cloning, expression, and purification difficulties are particularly acute. The DEBS proteins from native culture could be identified by our CP-labeling Western blot techniques following incubation of cell lysate with Sfp and biotinylated CoA analogs (FIG. 21B). Here, DEBS1, DEBS2, and DEBS3, with molecular weights of 365.1, 374.5, and 331.5 kDa, respectively, ran as one band and were readily visualized in amounts below the detection limit of Coomassie visualization. A faint band seen at 150 kDa was a native biotin-labeled protein. Tagging efficiency remained only modest, and Western blot visualization proved to be acutely sensitive to the media for culture growth, the timing of cell harvesting, and the conditions of cell lysate preparation. Clearly, natural PPTases in producer organisms effectively modify the majority of available CP domains. Methods to revert or inhibit 4′-phosphopantetheinylation are being investigated to alleviate this issue.

### EXAMPLE 19

Affinity Chromatography

The above labeling methods could be transferred to affinity purification techniques in order to isolate synthases with carrier protein domains. Cuatrecasas, P., et al., J. Biol. Chem., 245: 3059-3055, 1970. Cell lysate with apo-VibB was incubated with Sfp and biotinylated CoA analogs and the mixture was run over a small column loaded with streptavidin-linked-agarose resin. Following washing, the resin was boiled to release biotin-bound protein. A sample was subjected to SDS-PAGE and a Western blot against streptavidin-phosphatase conjugate. Both the Coomassie-stained gel and the Western blot indicated that biotin-tagged cypto-VibB was successfully purified with biotin affinity chromatography.

Due to denaturation involved in the recovery from streptavidin/biotin affinity purification, the non-denaturing conditions given by the affinity between carbohydrate-tagged proteins (i.e., α-mannosylated proteins) and lectin linked-agarose resins (i.e., concanavalin A) was examined. Maleimide 1f (Table 1) was coupled to CoA to yield α-mannosidylated CoA analog. Ahmed, M. S., et al., Membrane Biochem., 3: 329-340, 1980. Incubating the α-mannosidylated CoA analog with cell lysate of E. coli producing recombinant VibB and exogenous Sfp, crypto-VibB was produced with α-mannosyl groups. An aliquot of this mixture was bound to concanavalin A-linked agarose and washed on a small column. Bound protein was eluted off the agarose with a gradient of glucose, and the purified protein was identified with SDS-PAGE to yield a single band that was identified by Western blotting against concanavalin A-peroxidase conjugate. This protocol therefore produced pure, non-denatured α-mannosylated crypto-VibB In the purified form, crypto-VibB is not catalytically active, as the 4′-phosphopantetheinyl thiol remains covalently bound to the reporter. However, other domains associated with the CP domain (for example, condensation, adenylation, and thioesterase domains in NRP synthases) retain activity, and functional studies on these domains remains viable. In conclusion, this technique can be used with a variety of affinity methods and will further allow functional characterization of other active domains within a purified synthase. Methods are being investigating by which to reconstitute activity from labeled carrier protein domains.

A robust system for specifically labeling carrier protein domains within PK and NRP synthases has been demonstrated. This technique provides access to the fluorescent labeling, Western blotting, and affinity purification of carrier proteins. These tools provide a means to screen, quantify, and isolate these enzymes. Given the size and complexity of multi-domain biosynthetic systems, techniques are needed to quantify expression, solubility, folding, activity, and post-translational modification of these proteins in heterologous expression systems. These techniques can serve as diagnostic tools in metabolic engineering and combinatorial biosynthesis programs and can also be applicable in the search for natural product biosynthetic machinery in novel producer strains.

### EXAMPLE 20

Coenzyme A Analog Preparation

Six different maleimides are displayed in Table 1. Fluorescent maleimides 1a-c (Molecular Probes, Seattle, Wash.), 1d (Sigma-Aldrich, Milwaukee, Wis.) and 1e (Quanta Biodesign, Powell, Ohio) were obtained. α-Mannoside 1f was prepared according to Ahmed, et al. Cuatrecasas, P., et al., J. Biol. Chem., 245: 3059-3055, 1970. An aliquot of maleimide 1 (4.8 μL of 25 mg/mL solution of 1a in DMSO, 13.5 μL of a 10 mg/mL solution of 1b in DMSO, 8.7 μL of a 10 mg/mL solution of 1c in DMSO, 5.2 μL of a 25 mg/mL solution of 1d in DMSO, 6.0 μL of a 25 mg/mL solution of 1e in DMSO, and 4.0 μL of a 25 mg/mL solution of 1f in DMSO) was added to coenzyme A disodium salt (300 μg, 0.37 μmol) in 1.9 mL MES acetate and 100 mM Mg(OAc)₂ at pH 6.0 containing 300 μL DMSO. The resulting solution was vortexed briefly, cooled for 30 min at 0° C. and warmed at room temp for 10 min. CoA-maleimide formation was followed by thin layer chromatography (butanol/HOAc/water, 5:2:4). Extraction of the completed reaction with ethyl acetate (3×10 mL) was effective in removing excess 1a and 1c; the other maleimides were removed using scavenger resins N-linked 3-thiopropanoic acid PL-PEGA (Polymer Laboratories, Amherst, Mass.) or PS-thiophenol (Argonaut, Forester City, Calif.). This procedure provided stock solutions containing 100-125 μM modified CoA analogs from 1a-f.s.

### EXAMPLE 21

Carrier Protein Labeling Procedure

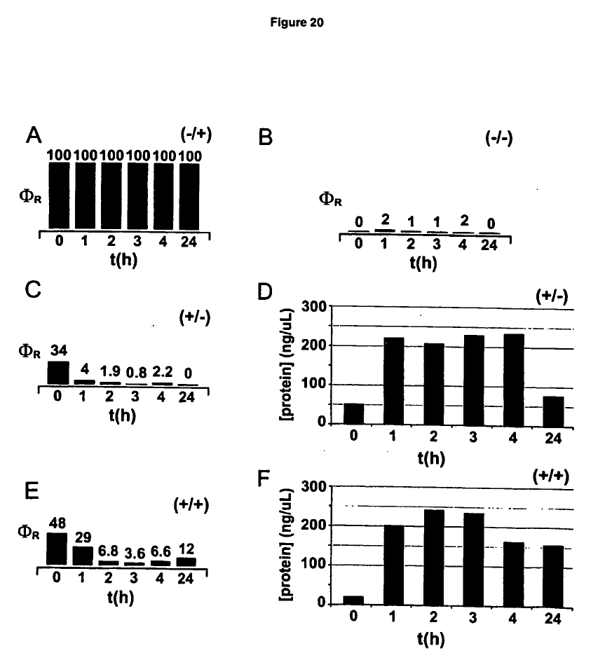

One Liter of E. Coli BL21 (DE3) cells induced to express recombinant VibB, FrenACP, OtcACP, and TcmACP, each in pET22b vectors (Novagen, Madison, Wis.), were pelleted, resuspended, and lysed by sonication in 30 mL 0.1 M Tris-Cl pH 8.0 with 1% glycerol in the presence of 500 μL of a 10 mM protease inhibitor cocktail containing bestatin, pepstatin A, E-64, and phosphoramidon (Sigma-Aldrich) and sonicated by pulsing for 5 minutes on ice. Alternatively, a lysozyme digestion was used in which the pellet was resuspended in lysis buffer A (20 M Na₂HPO₄ pH 7.8, 500 mM NaCl, 1 mg/mL lysozyme) and cooled on ice, and lysis buffer B (5% Triton X 100, 20 U/ml DNAse I, 20 U/mL RNAse) to 20% volume was then added. A 40 μL aliquot of a 100 μM solution of a Bodipy FL CoA analog was added 200 μL of cell lysate containing overexpressed protein and 1 μL of a 34 mg/mL solution of purified Sfp, and the reaction was incubated at room temperature for 30 minutes in darkness. When required (FIG. 20), recombinant His-tagged carrier proteins were purified by nickel chromatography using Ni—NTA His-Bind Resin (Novagen) according to manufacturer prodecure and dialyzed against 0.1 M Tris-HCl, pH 8.4 with 1% glycerol. Proteins were precipitated with 10% trichloroacetic acid, pelleted, washed, and the pellet was resuspended in 1:1 mixture of 1.0 M Tris-HCl pH 6.8 and 2× SDS-PAGE sample buffer (100 mM Tris-HCl pH 6.8, 4% SDS, 20% glycerol, 0.02% bromophenol blue). The samples were in boiled for 5 minutes and separated using SDS-PAGE electrophoresis on a 12% Tris-Glycine. Tagged proteins were visualized by trans-illumination (γ=365 nm) and the resulting images captured with CCD camera using a 475 nm cutoff filter. Protein concentration was determined using the Bradford method with bovine serum albumin (Sigma-Aldrich) as a standard.

### EXAMPLE 22

Expression Time Course Studies

Cultures of BL21(DE3) with TcmACP and (±) Sfp were grown in 100 mL of LB medium supplemented with the corresponding antibiotics. Gene expression was induced at OD₍₅₉₀₎=0.6 with 1 mM ITPG. At the indicated time points, 15 mL aliquots were removed from the culture, cooled, and pelleted Pellets were lysed and spun, and 250 μL of lysate was added to 100 μL of a 100 μM solution of a Bodipy FL CoA analog The reaction initiated with (±) 1 μL (30 mg/mL) purified Sfp or 1 μL water. Reactions were incubated in the dark at room temperature for 30 min, and the proteins were purified by nickel chromatography with EDTA elution. 150 μL of the eluates were analyzed for fluorescent intensity (excitation, γ=492 nm; emission, γ=535 nm).

### EXAMPLE 23

Western Blotting

Following SDS-PAGE separation of cell lysate using reporter a biotinylated CoA analog, the gel was electrophoretically transferred to nitrocellulose. Blots were incubated with 5% milk in TBST for 30 minutes at room temperature with shaking. The blots were then assayed with 10 mL of 5% milk in TBST solution containing either 10 μL of 25 mg/mL concanavalin A-peroxidase (Sigma-Aldrich) or 10 μL of 25 mg/mL streptavidin-alkaline phosphatase conjugate (Pierce Chemical Co., Rockford, Ill.). Following incubation at room temperature for 1 h, the blot was washed 3× for 10 minutes with 20 mL of TBST at room temperature and incubated in 2 mL of either peroxidase substrate solution (Sigma-Aldrich) containing 0.6 mg/ml 3,3-diaminobenzidine tetrahydrochloride in 50 mM Tris (pH 7.6) and 5 μL 30% hydrogen peroxide or alkaline-phosphatase substrate solution containing 0.15 mg/mL BCIP, 0.30 mg/mL NBT, 100 mM Tris pH 9.0, 5 mM MgCl₂ pH 9.5 (Sigma-Aldrich).

For DEBS Western, Saccharopolyspora erythraea was grown according to Caffrey, et al., in minimal medium (0.2 M sucrose, 20 mM succinic acid, 20 mM K₂SO₄ (pH 6.6), 5 mM Mg₂SO₄, 100 mM KNO₃, 2 mL/L trace element solution). Caffrey P., et al., FEBS Lett., 304: 225-8, 1992. 100 ml 1 L of culture was inoculated with a 100 mL 3-day growth and allowed to grow for four days. Cells were centrifuged and resuspended in 50 mL resuspension buffer: 50 mM Tris-Cl pH 7.5, 50% (v/v) glycerol, 2 mM DTT, 0.4 mM PMSF, 100 μg/mL DNAse, and 20 μg/mL RNAse, and 1 μL/mL bacterial protease inhibitor coctail (Sigma-Aldrich). The suspension was sonicated 10×30 seconds, ultracentrifuged 2 hrs. at 40 k×g, and the supernatant was labeled with Sfp and 3e. The reaction product was separated by a 3-8% Tris-acetate SDS-PAGE. The resulting gel was blotted onto nitrocellulose and developed as above with streptavidin-alkaline phosphatase conjugate and BCIP/NBT.

### EXAMPLE 24

Affinity Chromatography

Following cell lysis, 200 μL supernatant was combined with 40 μL of either a biotinylated CoA analog or a α-mannosidylated CoA analog and 1 μL of 11 mg/mL purified Sfp and allowed to react for 30 min at room temp in the dark. For biotinylated CoA analogs, 20 μL of agarose-immobilized streptavidin (4 mg/mL streptavidin on 4% beaded agarose, Sigma-Aldrich) was added, and the samples were and incubated at 4° C. for 1 hour with constant vigorous shaking. After centrifugation, the supernatant was decanted and the samples were washed 3× with a solution containing 100 mM Tris-HCl pH 8.4 and 1% SDS in water. After washing, the samples were boiled in 50 μL 1× SDS sample buffer for 10 min, centrifuged, and the supernatant run on a 12% Tris-Glycine SDS-PAGE gel and analyzed by Western blot. For the α-mannosidylated CoA analog, 20 μL of agarose-immobilized concanavalin A (4 mg/mL Jack bean concanavalin on 4% beaded agarose, Sigma-Aldrich) was added with binding buffer (1.3 mM CaCl₂, 1.0 mM MgCl₂, 1 mM MnSO₄, 10 mM KCl, 10 mM Tris pH 6.7) and incubated at 4° C. for 12 h. The beads were washed with binding buffer with 1% Triton X-100, and labeled carrier proteins were eluted with binding buffer with 20 mM glycine, 60 mM NaCl, 1% Triton X-100 and a gradient of 0-500 mM glucose. The elutate was run on a 12% Tris-Glycine SDS-PAGE gel and analyzed by Western blot.

### EXAMPLE 25

Uptake of Radiolabeled Coenzyme A Thioesters

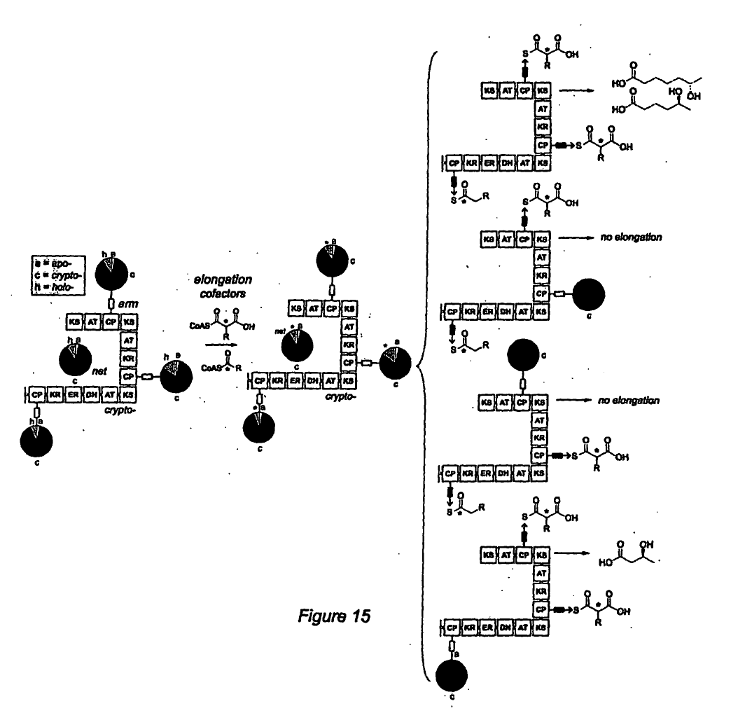

Of the first in vitro experiments, size and module number of the isolated synthases will be the first information gained. This experiment is demonstrated by digestion mapping with trypsin, 50 mM MES acetate or appropriate buffer. Partial digestion will be performed and protease profiler kits (Sigma) will be used to screen for alternative proteases. An aliquot of purified protein will be converted to the crypto-synthase, which can be visualized as a fluorescent band on a gel. Several synthases may be present from a single purification step, in which case they may be isolated by size exclusion or ion exchange chromatography. Once purified, the crypto-synthase will be digested with either trypsin, elastase, endoproteinase Glu-C, or endoproteinase Arg-C at various molar ratios for various lengths of time, and the resulting fragmentation patterns will yield dissected versions F1-F6 of the whole. When visualized via SDS-PAGE fluorescence/Coomassie analysis or HPLC, the protein fragment products may be analyzed to determine the number and location of individual CP domains (Aparicio 1994, Tsukamoto 1996). FIG. 15 shows a small number of fragments for demonstration purposes. The proteolytic cleavage of large proteins (>100 kD) often results in >100 peptides. For instance, the trypsin digest of modules 1 and 2 in the DEBS1 synthase leads to 304 fragments. HPLC, gel, and affinity-based methods can be used to isolate the fluorescent peptides F2, F4, F6 from within this mixture. By varying the protease in a series of parallel reactions, a broader view of the synthase identity and makeup may be assembled. These analyses add an extra element of fluorescence labeling to well-established protein chemistry techniques (Rosenberg 2002). Each of these proteolytic fragments are purified (SDS-PAGE, HPLC) and further analyzed for amino acid sequence.

With isolated amphidinium synthases in hand we can begin to ask basic biochemical questions about the biosynthetic mechanisms in the synthase, including the identity of individual modules. As described in the Background and Significance, one of the most topical questions in dinoflagellate biosynthesis is the nature of non-canonical carbon backbone linkages, which have been identified through isotope feeding experiments (Min 1989, Kobayashi 2004). One of the most compelling explanations of this phenomenon is the loading of alternate monomers, such as α-ketoglutaryl-CoA and succinyl-CoA (Chou 1987). Based on our three-module example repeated throughout this proposal, we would expect module three to load one of these alternate substrates. In order to probe this phenomenon, the synthase will be probed for uptake by incubation with radiolabeled a series of possible CoA-monomers, precipitated with trichloroacetic acid, and analyzed by scintillation or radioisotope SDS-PAGE (Aparicio 1994). The various radiolabeled acyl-CoA substrates to be attempted for module three loading will include malonyl-CoA, methylmalonyl-CoA, acetyl-CoA, succinyl-CoA, and α-ketoglutaryl-CoA. Of these, only α-ketoglutaryl-CoA is not available commercially and would need to be generated by enzymatic conversion of radiolabeled ketoglutarate and acetaldehyde dehydrogenase (Hosoi 1979). This general synthetic method can additionally be used to generate a variety of other CoA derivatives, which will be used as potential alternate substrates.

Significant advantages can be seen when both crypto-labeling and isotope uptake procedures are combined. We demonstrate these benefits with two different studies. The first probes intermediates in the pathway, and the second identifies monomer uptake by individual modules.

Current research into the mechanisms of modular synthases has focused on the identity of intermediates along individual pathways. Walsh and Kelleher have recently demonstrated a means to visualize intermediates of epothilone biosynthesis through tandem protease digestion and LCMS analysis to isolate and identify pathway intermediaries (Hicks 2004). As shown in FIG. 15, we propose an alternative approach to identifying such intermediates through the use of partial crypto-modification within a synthase. Because crypto-CP domains are catalytically blocked, biosynthesis of polyketides being processed down the synthase assembly line will be halted at these crypto-domains. Because partial crypto-modification will yield a distribution of labeling on the CP domains within each synthase, each intermediate moving along the synthase will be halted when it reaches a blocked crypto-CP. If during incubation isotope labeled CoA monomers are added to the reaction mixture, they will be taken up into the intermediates. The ketide (or peptide) intermediates may then be hydrolyzed from their thioester linkages after incubation by treatment with base and visualized by TLC. Structure elucidation of these intermediates may also be performed by using of stable isotope-labeled (¹³C) CoA-monomers in the reaction mixture, and the resulting intermediates may be elucidated by standard polyketide identity methods, including NMR and MS techniques (Geismann 1973). As shown in FIG. 15, the uptake of radioisotopically labeled Coenzyme A thioesters (e.g., malonyl-[2-¹⁴C]-CoA) will be examined using synthases-partially modified in the crypto-state with fluorescent dyes. Comparative analysis will be used to determine the relative uptake of isotopic labels as compared to the fluorescence from modified carrier protein domains. The formation of thioesters at each CP domain combines to provide a net uptake of radiolabel. As the radiolabeled crypto-synthase contains a distribution of fluorescent modifications, the processing of radiolabel reflects this collection of states. A selection of states and the resulting radioactive ketides synthesized by these states has been provided to illustrate the outcome of this experiment. The addition of sets of labeled Coenzyme A thioesters can be used to probe the substrate selectivity of the synthase.

### EXAMPLE 26

Uptake of Radioisotopically Labeled CoA Monomers Within Proteolytic Digests of Fluorescently Tagged Crypto-Synthase.

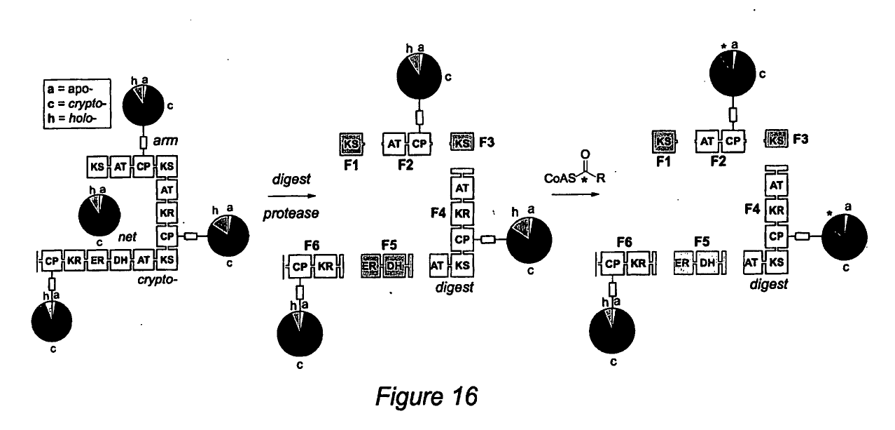

With a combination of the three techniques described in this section, crypto-labeling, isotope monomer loading, and proteolysis mapping, specific module identity can be gleaned from an isolated synthase. Proteolysis and radiolabeled monomer loading experiments such as these are well established in the literature for polyketide synthases (Aparicio 1994), and now we apply these techniques with the additional information given by crypto-CP florescence. FIG. 16 demonstrates this experiment. The synthase will first be partially labeled as the fluorescent crypto-form, where a percentage of each CP domain remains in apo- or crypto-form. Subsequent digest by proteases will cleave the synthase into fragments F2, F4, F6and these fragments can be used for radiolabeled monomer uptake experiments. Different radiolabeled CoA-monomers will be added to the proteolytic product in parallel experiments, and reactions will be separated by SDS-PAGE. These gels may be visualized by fluorescence and by phosphoimagry, and a comparison of the two images with the Coomassie stained gel will indicate which fragments contain CP domains and which CoA-monomer is loaded onto which CP domain. These experiments may be repeated with several different proteases in order to collect a full view of the synthase architecture. Non-radiolabeled versions of these proteolytic products may also be excised and sequenced (Smith 2003). FIG. 16 shows iptake in proteolytic digests of fluorescently tagged crypto-synthase. The uptake of isotopically labeled CoA-monomers within proteolytic fragments from the digests of amphidinolide synthase. Each protein fragment carrying an active AT-CP pair (F2 and F4-64) will load its cognate monomer onto the crypto-CP domain. Comparison of SDS-PAGE gels by fluorescence and phosphoimaging will verify which fragments contain CP domains and monomer identity loaded on each. Varying protease and CoA-monomer yields a broad description of synthase CP domain identity.

With purified synthase in hand, in vitro reconstitution of amphidinolide biosynthesis exists as a realistic goal. Cell-free reconstitution of polyketide synthases has been documented in the literature (Spencer 1992, Pieper 1995, Wiesmann 1995), although the difficulty to isolate whole synthases has frustrated many attempts at successful in vitro activity. With the techniques identified in this proposal for synthase purification and active crypto-synthase reconstitution, isolation and activity problems should be alleviated. A complete understanding of CoA monomer identity will be necessary before cell-free activity may be conducted, and we anticipate that sections D.3.a-c should clarify these concerns. Other issues to resolve are cofactor requirements, pH optimization, reducing potential, and overall enzyme stability. Many of these concerns will have been addressed in the previous sections, and the rest of these conditions will be replicated from literature examples of cell-free polyketide synthase activity (Spencer 1992, Pieper 1995, Wiesmann 1995). Successful activity will be probed with TLC and MS analysis. Should these methods prove too insensitive, radiolabeled monomer analogs will be used to amplify the signal.

### EXAMPLE 27

Serially Addressable Fusion Protein-Tag (SAFP-TAG) Fusion Proteins

Compositions and methods of the present invention can be used to construct the Serially Addressable Fusion Protein-Tag (SAFP-TAG) fusion protein system. A fusion protein system can be created for these studies. One of the smallest polyketide carrier proteins proteins, frnN, the frenolicin acyl CP from S. roseofulvus, contains 83 amino acids and demonstrates robust expression in E. coli from a C-terminal histidine-tagged expression vector (pET22). We will modify construct pET22-frnN with the at the 3′-end of the gene to convert it to a C-terminal fusion vector pDESTc-frnN compatible with the Gateway cloning system (Invitrogen, San Diego, Calif.). To create the N-terminal fusion, we will subclone the gene to include the natural stop codon back into pET22 and modify the construct at the 5′-end of the gene to create pDESTn-frnN. These two destination vectors will then be used to create a variety of fusion proteins from both eukaryotic and prokaryotic genes.

Modifying enzymes can be screened for optimal labeling kinetics. Over 200 PPTase sequences have been annotated Genbank, and thousands more are accessible from NRP and PK expressing organisms. We will clone and express 15-20 of these PPTases from several bacterial and filamentous fungal species. Literature precedent has demonstrated that some PPTases display selective recognition of CP domains. For example, while it is well established that the E. coli PPTase EntD, responsible for modifying EntB, it is not sufficient not load other secondary metabolic CP domains. For our purposes, it is important to choose an optimal CP-PPTase pair that demonstrate specificity for each other and accept CoA derivatives but do not label other proteins in the E. coli cell lysate.

Organisms with PPTase sequences in Genbank will be obtained from the American Type Culture Collection (ATCC), grown with appropriate conditions, and genomic DNA will be isolated through a general benzyl chloride procedure PCR amplification, cloning, and expression will be followed by PPTase activity studies involving fluorescent and chemical reporters of various sizes and chemical attributes. After an activity comparison of various PPTases, we will be in a position to choose the optimal enzyme system for fusion protein labeling. The chosen enzyme will then be applied to screen alternate affinity label attachment.

Affinity labels can be screened for manipulation of tagged fusion proteins. Several fluorescent and affinity reporter molecules have been used. However, almost any biocompatible molecule can be attached to the CP domain in the compositions and methods of the present invention. A variety of maleimide-reporter systems will be synthesized for visualization and affinity uses. These will include, but are not limited to, peptide tags, such as poly-histidine and FLAG-tag; carbohydrate tags, such as cellulose and sialyl-Lewis^{x}; metal-tags, such as chelated mercury and nickel; DNA tags containing both single- and double-stranded fusions; lipid tags, including myristate, palmitate, and other bioactive fatty acids; radioactive tags with ³H, ³⁵S, ³²P, or ¹⁴C labeled molecules.

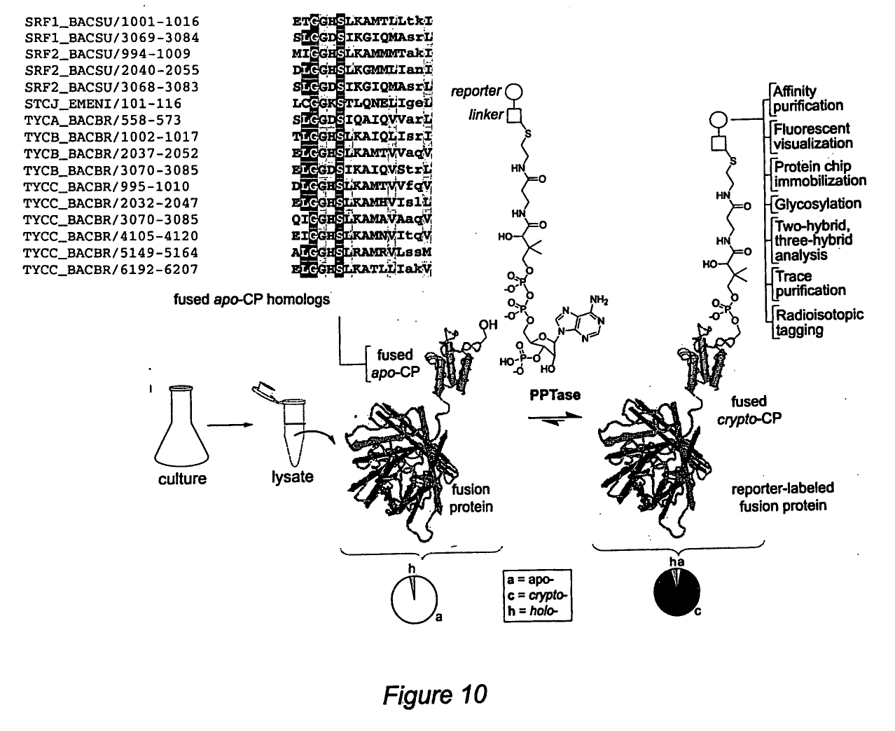

FIG. 10 shows an application of the composition and method to tag fusion molecules with an SAFP-TAG.

“Fused apo-CP homologs” refers to known CP domains having a consensus sequence within which the post-translational modification takes place. A fusion protein of the present invention can contain the consensus amino acid sequence or a homologous sequence thereof. The fusion partner can be as short as 13 amino acids, but it is considered a phosphopantetheinylation site if it has the consensus pattern. The consensus sequence is the following: [DEQGSTALMKRH]-[LIVMFYSTAC]-[GNQ]-[LIVMFYAG]-[DNEKHS]-S-[LIVMST]-{PCFY}-[STAGCPQLIVMF]-[LIVMATN]-[DENQGTAKRHLM]-[LIVMWSTA]-[LIVGSTACR]-x(2)-[LIVMFA]; wherein S is the pantetheine attachment site. Concise Encyclopedia Biochemistry, Second Edition, Walter de Gruyter, Berlin New-York (1988); Pugh E. L., et al., J. Biol. Chem. 240: 4727-4733, 1965; Witkowski A., et al. Eur. J. Biochem. 198: 571-579, 1991; http://us.expasy.org/cgi-bin/nicedoc.pl?PDOC00012.

The pattern rules are as follows. The PA (PAttern) lines contains the definition of a PROSITE pattern. The patterns are described using the following conventions: The standard IUPAC one-letter codes for the amino acids are used. The symbol ‘x’ is used for a position where any amino acid is accepted. Ambiguities are indicated by listing the acceptable amino acids for a given position, between square parentheses ‘[ ]’. For example: [ALT] stands for Ala or Leu or Thr. Ambiguities are also indicated by listing between a pair of curly brackets ‘{ }’ the amino acids that are not accepted at a given position. For example: {AM} stands for any amino acid except Ala and Met. Each element in a pattern is separated from its neighbor by a ‘-’. Repetition of an element of the pattern can be indicated by following that element with a numerical value or a numerical range between parenthesis. For example: x(3) corresponds to x-x-x, x(2,4) corresponds to x-x or x-x-x or x-x-x-x. When a pattern is restricted to either the N— or C-terminal of a sequence, that pattern either starts with a ‘<’ symbol or respectively ends with a ‘>’ symbol. In some rare cases (e.g. PS00267 or PS00539), ‘>’ can also occur inside square brackets for the C-terminal element. ‘F-[GSTV]-P-R-L-[G>]’ means that either ‘F-[GSTV]-P-R-L-G’ or ‘F-[GSTV]-P-R-L>’ are considered. A period ends the pattern.

All publications and patent applications cited in this specification are herein incorporated by reference in their entirety for all purposes as if each individual publication or patent application were specifically and individually indicated to be incorporated by reference for all purposes.

Although the foregoing invention has been described in some detail by way of illustration and example for purposes of clarity of understanding, it will be readily apparent to one of ordinary skill in the art in light of the teachings of this invention that certain changes and modifications may be made thereto without departing from the spirit or scope of the appended claims.

1. A method for detecting a protein of interest comprising:
contacting a coenzyme with a synthetic appendage label,
contacting a carrier protein domain with the protein of interest to form a carrier protein (CP) domain-protein of interest (POI) complex,
contacting the carrier protein (CP) domain-protein of interest (POI) complex with the labeled coenzyme to form a labeled coenzyme-carrier protein (CP) domain-protein of interest (POI) complex, and
detecting the labeled carrier protein domain to detect the protein of interest.

2. The method of claim 1 wherein the CP domain is a biosynthetic enzyme carrier protein domain.

3. The method of claim 2 wherein the protein of interest is a biosynthetic enzyme.

4. The method of claim 2 wherein the carrier protein domain is a polyketide (PK) synthase carrier protein domain, a non-ribosomal peptide (NRP) synthase carrier protein domain, or a fatty acid (FA) synthase carrier protein domain.

5. The method of claim 4 wherein the polyketide (PK) synthase carrier protein domain comprises at least one domain with acyl carrier protein (ACP) activity.

6. The method of claim 4 wherein the non-ribosomal peptide (NRP) synthase carrier protein domain comprises at least one domain with peptidyl carrier protein (PCP), aryl carrier protein (ArCP) and/or acyl carrier protein (ACP) activity.

7. The method of claim 4 wherein the fatty acid (FA) synthase carrier protein domain comprises at least one domain with acyl carrier protein (ACP) activity.

8. The method of claim 3 wherein the biosynthetic enzyme is a hybrid between a FA synthase, PK synthase, and/or NRP synthase and further comprises at least one domain with acyl carrier protein (ACP) and/or aryl carrier protein (ArCP) activity.

9. The method of claim 3, further comprising digesting the biosynthetic enzyme with a protease.

10. The method of claim 9, wherein the synthetic appendage label further comprises a linker and a reporter.

11. The method of claim 9, further comprising contacting the labeled coenzyme-carrier protein (CP) domain-protein of interest (POI) complex with a radioactively-labeled coenzyme to form a radioactively labeled coenzyme-carrier protein (CP) domain-protein of interest (POI) complex.

12. The method of claim 1, further comprising contacting the labeled coenzyme-carrier protein (CP) domain-protein of interest (POI) complex with a radioactively-labeled coenzyme to form a radioactively labeled coenzyme-carrier protein (CP) domain-protein of interest (POI) complex.

13. The method of claim 1, wherein said contacting the carrier protein (CP) domain with the protein of interest (POI) further comprises synthesizing a CP domain-POI fusion protein to form a carrier protein (CP) domain-protein of interest (POI) complex.

14. The method of claim 13, wherein the carrier protein (CP) domain further comprises an amino acid consensus sequence, [DEQGSTALMKRH]-[LIVMFYSTAC]-[GNQ]-[LIVMFYAG]-[DNEKHS]-S-[LIVMST]-{PCFY}-[STAGCPQLIVMF]-[LIVMATN]-[DENQGTAKRHLM]-[LIVMWSTA]-[LIVGSTACR]-(x2)-[LIVMFA].

15. The method of claim 1 wherein the labeled coenzyme-CP domain-POI complex further comprises coenzyme A (CoA) or a derivative thereof.

16. The method of claim 1 further comprising contacting the CP domain-POI complex and the labeled coenzyme with a phosphotransferase enzyme to form a labeled coenzyme-CP domain-POI complex.

17. The method of claim 16 wherein the phosphotransferase enzyme is a 4′-phosphopantetheinyl transferase.

18. The method of claim 10 wherein the reporter is an affinity reporter, a colored reporter, a fluorescent reporter, a magnetic reporter, a radioisotopic reporter, a peptide reporter, a metal reporter, a nucleic acid reporter, a lipid reporter, a glycosylation reporter, or a reactive reporter.

19. The method of claim 18, wherein the synthetic appendage label further comprises a protein chip immobilization label, a two-hybrid or three-hybrid analysis label, or a trace purification label.

20. The method of claim 10, wherein the reporter is a precursor to an affinity reporter, a colored reporter, a fluorescent reporter, a magnetic reporter, a radioisotopic reporter, a peptide reporter, a metal reporter, a nucleic acid reporter, a lipid reporter, a glycosylation reporter, or a reactive reporter.

21. The method of claim 1 further comprising detecting or modulating a function of label by interaction with a secondary molecule.

22. The method of claim 21 wherein the secondary molecule is a carbohydrate, a protein, a peptide, an oligonucleotide, or a synthetic receptor.

23. The method of claim 3, further comprising assembling libraries of biosynthetic enzymes, coenzymes and synthetic appendage labels,
contacting individual units of biosynthetic enzymes, coenzymes and synthetic appendage labels from libraries of POIs, coenzymes and synthetic appendage labels, and
detecting transfer of synthetic appendage label from coenzyme to carrier protein of the biosynthetic enzyme, wherein specificity of the transfer detects the biosynthetic enzyme.

24. The method of claim 23 wherein the individual units from libraries of coenzymes are spatially-addressed on a three dimensional object.

25. The method of claim 23 wherein the individual units from libraries of enzymes are spatially-addressed on a three dimensional object.

26. The method of claim 23 wherein the individual units from libraries of labels are spatially-addressed on a three dimensional object.

27. The method of claim 23 wherein the individual units from libraries of coenzymes and libraries of enzymes are spatially-addressed on a three dimensional object.

28. The method of claim 23 wherein the individual units from libraries of coenzymes and labels are spatially-addressed on a three dimensional object.

29. The method of claim 23 wherein the individual units from libraries of coenzymes, labels and enzymes are spatially-addressed on a three dimensional object.

30. The method of claim 1 further comprising identifying the biosynthetic enzyme within a cell culture.

31. The method of claim 1 further comprising identifying the biosynthetic enzyme by molecular weight.

32. The method of claim 31 wherein the enzyme molecular weight is determined by a technique selected from gel electrophoresis, affinity chromatography or mass spectrometry.

33. The method of claim 1 further comprising identifying the protein of interest by nucleic acid or protein sequencing.

34. The method of claim 1 further comprising isolating the protein of interest.

35. The method of claim 1 further comprising assaying for the expression and/or activity of the protein of interest.

36. The method of claim 1 further comprising screening for proteins of interest.

37. The method of claim 35 further comprising quantifying the expression a given protein of interest or group of proteins of interest.

38. The method of claim 23, further comprising quantifying temporal events related to the expression a given protein of interest.

39. The method of claim 1 further comprising identifying a cell, cell-line, organism or class of organisms characterized by the marking of the protein of interest with the label.

40. The method of claim 39 further comprising determining a time of infection or a stage in a cell cycle or a stage in a life cycle.

41. The method of claim 39 further comprising determining a level of virulence of the organism.

42. The method of claim 3 further comprising identifying novel natural products from the biosynthetic enzyme.

43. The method of claim 3 further comprising screening for inhibitors of the biosynthetic pathways.

44. The method of claim 3 further comprising measuring individual responses of the biosynthetic enzyme to given conditions to identify the biosynthetic enzyme using a profiler.

45. The method of claim 1, further comprising removing chemically or enzymatically the product generated from the transfer of the synthetic appendage label.

46. The method of claim 45 further comprising removing the synthetic appendage label from the carrier protein domain by light.

47. The method of claim 45 further comprising removing the synthetic appendage label from the carrier protein domain by heat.

48. The method of claim 45 further comprising removing the synthetic appendage label from the carrier protein domain by a chemical reagent.

49. The method of claim 45 further comprising removing the synthetic appendage label from the carrier protein domain by an enzyme.

50. The method of claim 49 wherein the enzyme is an acyl carrier protein phosphodiesterase.

51. A microarray for identification of a protein of interest (POI) comprising:
a coenzyme linked to a synthetic appendage label,
a carrier protein domain contacting the labeled coenzyme and the POI to form a carrier protein-POI-coenzyme complex,
the synthetic appendage label transferred from the coenzyme to the carrier protein domain within the microarray, wherein the labeled carrier protein domain detects the POI.

52. The microarray of claim 51 further comprising individual units of enzymes derived from libraries of enzymes, coenzymes derived from libraries of coenzymes and synthetic appendage labels derived from libraries of synthetic appendage labels, wherein the individual units of enzymes, coenzymes and synthetic appendage labels are spatially addressed on a three dimensional object.

53. The microarray of claim 51 wherein the POI is a biosynthetic enzyme.

54. The microarray of claim 53 wherein the biosynthetic enzyme is selected from a polyketide (PK) synthase, a non-ribosomal peptide (NRP) synthase, or a fatty acid (FA) synthase.

55. The microarray of claim 54 wherein the polyketide (PK) synthase comprises at least one domain with acyl carrier protein (ACP) activity.

56. The microarray of claim 54 wherein the non-ribosomal peptide (NRP) synthase comprises at least one domain with peptidyl carrier protein (PCP), aryl carrier protein (ArCP) and/or acyl carrier protein (ACP) activity.

57. The microarray of claim 54 wherein the fatty acid (FA) synthase comprises at least one domain with acyl carrier protein (ACP) and/or aryl carrier protein (ArCP) activity.

58. The microarray of claim 51 wherein the biosynthetic enzyme comprises a hybrid between a FA synthase, PK synthase, and/or NRP synthase and further comprises at least one domain with acyl carrier protein (ACP) and/or aryl carrier protein (ArCP) activity.

59. The microarray of claim 51 wherein the carrier protein-enzyme-coenzyme complex further comprises coenzyme A (CoA) or a derivative thereof.

60. The microarray of claim 51 wherein the carrier protein-POI-coenzyme complex further comprises a phosphotransferase enzyme.

61. The microarray of claim 51 wherein the phosphotransferase enzyme is a 4′-phosphopantetheinyl transferase.

62. The microarray of claim 51 wherein the synthetic appendage label further comprises a linker and a reporter

63. The microarray of claim 62 wherein the reporter is an affinity reporter, a colored reporter, a fluorescent reporter, a magnetic reporter, a radioisotopic reporter, a peptide label, a metal reporter, a nucleic acid reporter, a lipid reporter, a glycosylation reporter, or a reactive reporter.

64. The microarray of claim 62 wherein the reporter is a precursor to an affinity reporter, a colored reporter, a fluorescent reporter, a magnetic reporter, a radioisotopic reporter, a peptide reporter, a metal reporter, a nucleic acid reporter, a lipid reporter, a glycosylation reporter, or a reactive reporter.

65. The microarray of claim 51 further comprising interaction with a secondary molecule to detect or modulate a function of the label.

66. The microarray of claim 65 wherein the secondary molecule is selected from a carbohydrate, a protein, a peptide, an oligonucleotide, or a synthetic receptor.

67. The microarray of claim 51, further comprising a profiler to measure individual responses of the biosynthetic enzyme to given conditions to identify the biosynthetic enzyme.

68. The microarray of claim 51, further comprising a product generated from the transfer of the synthetic appendage label to the carrier protein is removed chemically or enzymatically.

69. The method of claim 23 further comprising identifying the biosynthetic enzyme within a cell culture.

70. The method of claim 23 further comprising identifying the biosynthetic enzyme by molecular weight.

71. The method of claim 23 further comprising identifying the protein of interest by nucleic acid or protein sequencing.

72. The method of claim 23 further comprising isolating the protein of interest.

73. The method of claim 23 further comprising screening for proteins of interest.

74. The method of claim 23 further comprising identifying novel natural products from the biosynthetic enzyme.

75. The method of claim 23 further comprising screening for inhibitors of the biosynthetic pathways.

76. The method of claim 23 further comprising measuring individual responses of the biosynthetic enzyme to given conditions to identify the biosynthetic enzyme using a profiler.
